(19) United States
(12) Patent Application Publication
(10) Pub. No.: US 2024/0043787 A1
(43) Pub. Date: Feb. 8, 2024

(54) SYSTEMS AND METHODS FOR GENERATING MYCELIA GROWTH FROM SUBSTRATES

(71) Applicant: Ecovative Design LLC, Green Island, NY (US)

(72) Inventors: Hazel A. Robertson, Cambridge, NY (US); Molly Elizabeth Boutin, Troy, NY (US); Emmie Marie Knobloch, Rensselaer, NY (US); Gavin Reim McIntyre, Troy, NY (US); Peter James Mueller, Poestenkill, NY (US); Alex James Carlton, Troy, NY (US); Meghan Anne O’Brien, Halfmoon, NY (US)

(21) Appl. No.: 18/360,705

(22) Filed: Jul. 27, 2023

Related U.S. Application Data
(60) Provisional application No. 63/393,512, filed on Jul. 29, 2022.

(51) Int. Cl. C12N 1/14

(52) U.S. Cl. CPC C12N 1/14

(57) ABSTRACT

Methods are disclosed for generating aerial mycelium, such as mycological material comprising solely mycelia from depleted substrate or depleted and rejuvenated substrate. Alternatively, methods are disclosed for generating mycelia and mushrooms from depleted substrate or depleted and rejuvenated substrate. Alternative methods are disclosed for generating differentiated mycelium materials using depleted substrate or depleted and rejuvenated substrate. The mycelia products that are generated can be used in the food industry (e.g., as a meat analog) and in other industries, such as textiles, packaging, and others. The present invention provides systems and methods for generating mycelia that are repeatable and energy efficient, while providing consistently high quality and quantity mycelium-based products.

### CROSS-REFERENCE TO RELATED APPLICATIONS

This application is related to U.S. Provisional Patent Application No. 63/393,512, filed Jul. 29, 2022, entitled “SYSTEMS AND METHODS FOR GENERATING MYCELIA GROWTH FROM SUBSTRATES,” the disclosure of which is incorporated herein by reference in its entirety.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 C.F.R. § 1.57. This application claims the benefit of U.S. Provisional Patent Application No. 63/393,512, filed Jul. 29, 2022, entitled “SYSTEMS AND METHODS FOR GENERATING MYCELIA GROWTH FROM SUBSTRATES,” the disclosure of which is incorporated herein by reference in its entirety.

### FIELD

This application relates generally to mycelia suitable for use in the manufacture of mycelium-based food products, textile products, leather-like materials, petroleum-based product alternatives, foams, composites, and other products, and in particular, to systems and methods for generating mycelia for incorporation into said products.

### BACKGROUND

Environmentally friendly alternatives to traditional materials are in high demand, either in the food industry (e.g., meat analogs) or in non-food-related industries, such as textiles, packaging, construction, and other industries. Products made from fungal mycelia fill such demand. As a result, fungal mycelia are increasingly used as an efficient and biodegradable material across several industrial applications.

The growing demand for and relative novelty of mycelium-based products has resulted in a parallel need for fungal tissue growing methods that are reproducible and energy efficient. In general, there is a growing need for large-scale methods for growing mycelium-based products that yield quality tissue for cost-effective industrial applications.

### SUMMARY

For the purpose of summarizing the invention and the advantages achieved over the prior art, certain objects and advantages of the invention have been described herein. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

In a first aspect, a method of growing an aerial mycelium material is included. The method can include providing a depleted growth matrix, applying a mechanical force to the depleted growth matrix, and disrupting the depleted growth matrix with the mechanical force.

In various aspects, disrupting can include at least one of grinding, breaking, fracturing, cutting, perforating, fragmenting, and physically manipulating the depleted growth matrix.

In various aspects, the method can include growing extra-particle aerial mycelial growth from the depleted growth matrix.

In various aspects, the method can include rejuvenating at least a portion of the depleted growth matrix to form a rejuvenated growth matrix and growing extra-particle aerial mycelial growth from the rejuvenated growth matrix.

In some aspects, the method can include rejuvenating at least a portion of the depleted growth matrix to form a rejuvenated growth matrix and growing one or more mushroom fruiting bodies from the rejuvenated growth matrix.

In some aspects, the method can include detaching the extra-particle aerial mycelial growth from the rejuvenated growth matrix to form a separated aerial mycelium from the extra-particle aerial mycelial growth, wherein the extra-particle aerial mycelial growth and the separated aerial mycelium do not comprise a fruiting body.

In some aspects, rejuvenating can include at least one of sterilizing the depleted growth matrix, rehydrating the depleted growth matrix, and supplementing the depleted growth matrix with an additive(s).

In various aspects, rejuvenating can include supplementing the depleted growth matrix with the additive(s), wherein the additive(s) can include at least one of fresh fungal inoculum, fresh substrate, precolonized substrate, precolonized substrate which has been inoculated with fungal spawn and grown for up to 4 days before use, charcoal, and a nutrient source(s).

In some aspects, rejuvenating can include supplementing the depleted growth matrix with the additive(s) including fresh substrate at a mass ratio of between 1:3 and 3:1 depleted growth matrix to fresh substrate.

In another aspect, a method of growing an aerial mycelium material can include providing a depleted growth matrix, applying a mechanical force to the depleted growth matrix, disrupting the depleted growth matrix with the mechanical force, rejuvenating at least a portion of the depleted growth matrix to form a rejuvenated growth matrix, wherein rejuvenating includes supplementing the depleted growth matrix with an additive(s), and wherein the additive(s) include fresh substrate, and growing extra-particle aerial mycelial growth from the rejuvenated growth matrix.

In another aspect, a method of growing an aerial mycelium material can include providing a first extra-particle aerial mycelial growth and a first growth matrix, wherein the first extra-particle aerial mycelial growth extends from a surface of the first growth matrix, dividing the first extra-particle aerial mycelial growth from the first growth matrix along a first separation zone to form a first separated aerial mycelium and a depleted growth matrix, rejuvenating at least a portion of the depleted growth matrix to form a rejuvenated growth matrix, and growing a second extra-particle aerial mycelial growth from the rejuvenated growth matrix.

In some aspects, the method can further include applying a mechanical force to the depleted growth matrix and disrupting the depleted growth matrix with the mechanical force.

In various aspects, disrupting can include at least one of grinding, breaking, fracturing, cutting, perforating, fragmenting, and physically manipulating the depleted growth matrix.

In various aspects, dividing can include dividing the first extra-particle aerial mycelial growth from the first growth matrix along the first separation zone such that the depleted growth matrix includes a transitional layer of unused extra-particle aerial mycelial growth on an underlying remainder portion of the depleted growth matrix.

In some aspects, the method can further include removing the transitional layer from the underlying remainder portion of the depleted growth matrix prior to disrupting the depleted growth matrix and growing the second extra-particle aerial mycelial growth.

In various aspects, rejuvenating can include at least one of sterilizing the depleted growth matrix, rehydrating the depleted growth matrix, and supplementing the depleted growth matrix with an additive(s).

In some aspects, rejuvenating can include rehydrating the depleted growth matrix by raising the moisture content of the depleted growth matrix to between about 60% to about 75%, alternatively between about 62% to about 75%, or alternatively between about 65% to about 75%.

In various aspects, rehydrating can include gouging the substrate surface.

In various aspects, rejuvenating can include supplementing the depleted growth matrix with the additive(s), and wherein the additive(s) include at least one of fresh fungal inoculum, fresh substrate, precolonized substrate, precolonized substrate which has been inoculated with fungal spawn and grown for up to 4 days before use, charcoal, and a nutrient source(s).

In some aspects, rejuvenating can include supplementing the depleted growth matrix with the additive(s) comprising fresh substrate at a mass ratio of between 1:3 and 3:1 depleted growth matrix to fresh substrate.

In some aspects, the first growth matrix can include a first fungal inoculum, and the additive(s) can include the fresh fungal inoculum, wherein the first fungal inoculum and the fresh fungal inoculum are a different species relative to each other.

In various aspects, the method can include dividing the second extra-particle aerial mycelial growth from the rejuvenated growth matrix along a second separation zone to form a second separated aerial mycelium and a second depleted growth matrix, and cutting the second separated aerial mycelium in a transverse direction and across a width of the second separated aerial mycelium to form an aerial mycelium panel.

In another aspect, a method of making aerial mycelium panels can include growing a first extra-particle aerial mycelial growth from a first growth matrix such that the first extra-particle aerial mycelial growth extends from a surface of the first growth matrix, and without producing any fruiting bodies, harvesting a first aerial mycelium panel from the first extra-particle aerial mycelial growth, wherein harvesting can include dividing the first extra-particle aerial mycelial growth from the first growth matrix along a first separation zone to form a first separated aerial mycelium and a first depleted growth matrix and cutting the first separated aerial mycelium in a transverse direction and across a width of the first separated aerial mycelium to form the first aerial mycelium panel, applying a mechanical force to the first depleted growth matrix, disrupting the first depleted growth matrix with the mechanical force, rejuvenating at least a portion of the first depleted growth matrix to form a rejuvenated growth matrix, growing a second extra-particle aerial mycelial growth from the rejuvenated growth matrix such that the second extra-particle aerial mycelial growth extends from a surface of the rejuvenated growth matrix, and without producing any fruiting bodies, and harvesting a second aerial mycelium panel from the second extra-particle aerial mycelial growth, wherein harvesting comprises dividing the second extra-particle aerial mycelial growth from the rejuvenated growth matrix along a second separation zone to form a second separated aerial mycelium and a second depleted growth matrix and cutting the second separated aerial mycelium in a transverse direction and across a width of the second separated aerial mycelium to form the second aerial mycelium panel.

In some aspects, the method can further include rejuvenating at least a second portion of the first depleted growth matrix to form a second rejuvenated growth matrix and growing one or more mushroom fruiting bodies from the second rejuvenated growth matrix.

In some aspects, at least one of the first depleted growth matrix and the second depleted growth matrix can include a transitional layer of unused extra-particle aerial mycelial growth and an underlying remainder portion, wherein the method can further include removing the transitional layer from the underlying remainder portion.

In some aspects, the first separated aerial mycelium and the second separated aerial mycelium can be substantially the same with respect to one or more of the following physical characteristics: elasticity, tensile strength, rigidity, density, shear strength, texture, and compressive strength.

In another aspect, a method of growing either aerial mycelium material or mushrooms is included. The method can include providing a depleted growth matrix which has been depleted by a previous growth of mycelium therein and/or thereupon, applying a mechanical force to the depleted growth matrix, thereby disrupting the depleted growth matrix with the mechanical force, and growing either aerial mycelium or mushrooms upon the depleted growth matrix, wherein at least one of the following conditions is met: (i) the previous growth of mycelium comprises growth conditions preferentially favoring the growth of predominantly aerial mycelium, and (ii) the growing of either aerial mycelium or mushrooms upon the depleted growth matrix comprises growing aerial mycelium under growth conditions preferentially favoring the growth of predominantly aerial mycelium.

In some aspects, the method can further include rejuvenating the depleted growth matrix following the disrupting.

In some aspects, the method can further include containing the depleted growth matrix within packaging following the disrupting for later growth of mushrooms.

In various aspects, the previous growth of mycelium can have resulted in aerial mycelium without any mushroom fruiting bodies, wherein the growing of either aerial mycelium or mushrooms upon the depleted growth matrix can include growing one or more mushroom fruiting bodies.

In various aspects, the previous growth of mycelium can have resulted in mycelium with one or more mushroom fruiting bodies, wherein the growing of either aerial mycelium or mushrooms upon the depleted growth matrix can include growing aerial mycelium without any mushroom fruiting bodies.

In another aspect, a method of growing either aerial mycelium material or mushrooms can include forming a depleted growth matrix by at least one of (i) growing and harvesting aerial mycelium from an aerial mycelium growth matrix without producing any fruiting bodies and (ii) growing and harvesting one or more mushrooms bodies from a mushroom growth matrix, applying a mechanical force to the depleted growth matrix thereby disrupting the depleted growth matrix with the mechanical force, and growing either aerial mycelium or mushrooms upon the depleted growth matrix, wherein at least one of the following conditions is met: (a) the forming of the depleted growth matrix includes growing and harvesting aerial mycelium from the aerial mycelium growth matrix without producing any fruiting bodies, and (b) the growing either aerial mycelium or mushrooms upon the depleted growth matrix includes growing aerial mycelium upon the depleted growth matrix.

All of these embodiments are intended to be within the scope of the invention disclosed herein. These and other embodiments of the present invention will become readily apparent to those skilled in the art from the following detailed description of the alternative embodiments having reference to the attached figures, the invention not being limited to any particular alternative embodiment(s) disclosed.

### BRIEF DESCRIPTION OF THE DRAWINGS

The features and advantages of the methods and compositions described herein will become more fully apparent from the following description and appended claims, taken in conjunction with the accompanying drawings. These drawings depict only several embodiments in accordance with the disclosure and are not to be considered limiting of their scope. In the drawings, similar reference numbers or symbols typically identify similar components, unless context dictates otherwise. In some instances, the drawings may not be drawn to scale.

FIG. 1A illustrates an embodiment of a growth matrix suitable to support extra-particle aerial mycelial growth.

FIG. 1B illustrates an embodiment of extra-particle aerial mycelial growth extending from the growth matrix of FIG. 1A.

FIG. 1C illustrates an embodiment in which the extra-particle aerial mycelial growth and growth matrix in FIG. 1B have been divided to form a separated aerial mycelium and depleted growth matrix.

FIG. 1D illustrates an embodiment of the separated aerial mycelium material in FIG. 1C that has been cut to form an aerial mycelium panel.

FIG. 1E illustrates an embodiment of the depleted growth matrix in FIGS. 1C and 1D to which a mechanical force is being applied, to disrupt the depleted growth matrix.

FIG. 1F illustrates an embodiment of the depleted growth matrix in FIGS. 1D and 1E which is being rejuvenated.

FIG. 2 illustrates a flow diagram of an embodiment of a method of growing an aerial mycelium material.

FIG. 3 illustrates a flow diagram of another embodiment of a method of growing an aerial mycelium material.

FIG. 4 illustrates a flow diagram of an embodiment of a method of making aerial mycelium material panels.

FIG. 5 illustrates a flow diagram of an embodiment of growing aerial mycelium material.

FIG. 6 illustrates an embodiment of mycelia grown from depleted maple flakes.

FIGS. 7A and 7B illustrate embodiments of mycelia grown from rehydrated depleted substrate.

FIG. 8 illustrates an embodiment of little to no growth of mycelia from depleted substrate after the transitional layer has been removed.

FIG. 9 illustrates an embodiment of mycelia grown from depleted substrate after the transitional layer has been removed and the substrate has been rehydrated.

FIG. 10 illustrates an embodiment of mycelia grown from depleted substrate after the transitional layer has been removed and the substrate has been rehydrated and reinoculated.

FIG. 11 illustrates an embodiment of mycelia grown from depleted substrate which has been reground and reinoculated.

FIGS. 12A-C illustrate embodiments of mycelia grown from a block of depleted substrate which has been flipped upside down.

FIGS. 13A and 13B illustrate embodiments of mycelia grown from depleted substrate after the transitional layer has been removed.

FIGS. 14A-C illustrate embodiments of mycelia grown from depleted substrate which has been reground and rehydrated.

FIGS. 15A-C illustrate embodiments of mycelia grown from depleted substrate mixed with uninoculated substrate in a 1:1 ratio.

FIGS. 16A-C illustrate embodiments of mycelia grown from precolonized substrate.

FIGS. 17A-C illustrate embodiments of mycelia grown from depleted substrate mixed with precolonized substrate.

### DETAILED DESCRIPTION

U.S. Patent Application Publication No. 2015/0033620, International PCT Patent Application No. WO2019/099474A1, and U.S. Provisional Patent Application No. 63/341,965, the entirety of which are incorporated herein by reference thereto, except where inconsistent with the disclosure herein, describe systems and methods of growing and/or harvesting a mycological material and products resulting therefrom.

Yield efficiency is a major challenge facing large-scale methods of fungal mycelial production. Methods described herein are designed to reduce the quantity of input material (e.g., substrate) used to grow mycelial material while increasing the quantity and quality of fungal tissue produced. Methods described herein are also designed to be reproducible and energy-efficient, while also geared towards producing quality mycelium-based products in large quantities.

Described herein are embodiments of systems, apparatus, and methods to generate a mycelium material, such as a mycological material comprising aerial mycelium material. The embodiments can allow a growth matrix to provide a first growth therefrom and be rejuvenated and reused to provide a second aerial mycelium material growth therefrom with similar properties and qualities as the first aerial mycelium material growth. Alternatively, the embodiments can allow for a growth matrix to provide a first growth therefrom, and then can be used thereafter or rejuvenated thereafter to provide a second growth therefrom, wherein the second growth can result in the production of fruiting bodies (e.g., desired mushrooms from the mycelium).

The aerial mycelium material products that are generated can be used in the food industry (for example, as a meat-substitute product or meat analog, and one that may present to the consumer a product that offers the appearance and texture of traditional meat material, i.e., beef, pork, poultry and seafood), and in other industries, such as textiles, packaging, and others. As previously noted, the reused or rejuvenated growth matrix, which may include some quantity of mycelia (such as in the form of a transition layer) can additionally be used to later generate traditional mushroom harvests. It is an object of the present invention to provide mycelial growing methods and systems that are repeatable and energy efficient, while providing high quality and quantity mycelium-based products over a series of growth cycles. It is a further object of the present invention to provide growing methods that offer flexibility and adaptability such that the methods are capable of alternating between various growing step options (among several available, such as in sequence of steps, or overall steps utilized) so as to accommodate either varying product designs (with each design demonstrating differing desired product attributes) or to accommodate various manufacturing facility spaces or equipment availability. It is a further object of the present invention to utilize mycelium growth cycles to generate additional aerial mycelium material (without any or substantially any fruiting bodies or mushrooms, under growth conditions appropriate for fostering either primarily or exclusively prolific aerial mycelium production) as well as traditional mushrooms (under growth conditions appropriate for fostering the prolific growth of mushrooms, at the cost of aerial mycelium production) as may be desired. The growth conditions can include one or more of the following variables that may be selected or adjusted for the growth process: the gases (CO₂, N₂, O₂) (composition and concentration); relative humidity; atmospheric pressure airflow (velocity, direction, ‘horizontal’); temperature; mist/misting; (mean) mist deposition rate; duty cycle; mist cycle period; desiccation/drying; time (e.g., incubation time period); light (e.g., presence/absence/intensity/color); solutes (e.g., in mist) and conductivity (e.g., of mist).

The following discussion presents detailed descriptions of the several embodiments of the present disclosure shown in the Figures. These embodiments are not intended to be limiting, and modifications, variations, combinations, etc., are possible and within the scope of this disclosure.

### Definitions

The aerial mycelia of the present disclosure are growth products obtained from a growth matrix incubated for a period of time (i.e., an incubation time period) in a growth environment, as disclosed herein.

“Mycelium” as used herein refers to a connective network of fungal hyphae, with mycelia being the plural form of mycelium.

“Hyphae” as used herein refers to branched filament vegetative cellular structures that are interwoven to form mycelium.

“Substrate” as used herein refers to a material or surface thereof, from or on which an organism lives, grows and/or obtains its nourishment. In some embodiments, a substrate provides sufficient nutrition to the organism under target growth conditions such that the organism can live and grow without providing the organism a further source of nutrients; such a substrate may be referred to herein as a “nutritive substrate.” The composition of a substrate may be tailored to be beneficial for the organism selected to live, grow, and/or obtain its nourishment therefrom, or for the extent to which the organism is intended to grow. For example, a substrate composition for growing mushrooms (e.g., used to make a mushroom growth matrix) may be different from a substrate composition for growing aerial mycelium without a fruiting body (e.g., used to make an aerial mycelium growth matrix).

“Growth media” or “growth medium” as used herein refers to a matrix containing a substrate and an optional further source of nutrition that is the same as or different from the substrate, wherein the substrate, the nutrition source, or both are intended for fungal consumption to support mycelial growth.

“Growth matrix” as used herein refers to a matrix containing a growth medium and a fungus. In some embodiments, the fungus is provided as a fungal inoculum; thus, in such embodiments, the growth matrix comprises a fungal-inoculated growth medium. In other embodiments, the growth matrix comprises a colonized substrate.

“Depleted growth matrix” as used herein refers to a growth matrix which has supported some mycelial growth (such as, e.g., aerial mycelium and/or one or more mushroom fruiting bodies) and therefore is at least partially depleted of nutrients or other materials either after extra-particle aerial mycelial growth has been grown and divided from the growth matrix to form a separated mycelium material or alternatively, after mushrooms have been grown and separated from the growth material. Depleted growth matrix may also be referred to herein as depleted substrate. “Remainder substrate” may be referred to herein to specifically indicate depleted growth matrix which has supported targeted growth of one or more mushroom fruiting bodies (as part of a mushroom harvesting process) and is at least partially depleted of nutrients or other materials after the one or more mushroom fruiting bodies have been grown and divided from the growth matrix.

“Rejuvenated growth matrix” as used herein refers to a depleted growth matrix which has been further processed (e.g., chemically or mechanically) to improve its viability to support the targeted growth of an end product (e.g., extra-particle aerial mycelial growth, or alternatively, traditional mushrooms (fruiting bodies)).

“Inoculated substrate” as used herein refers to a substrate that has been inoculated with fungal inoculum. For example, an inoculated substrate can be formed by combining an uninoculated substrate with a fungal inoculum. An inoculated substrate can also be formed by combining an uninoculated substrate with a previously inoculated substrate. An inoculated substrate can also be formed by combining an inoculated substrate with a colonized substrate. The condition of “inoculated” may also be referred to herein as “fungal-inoculated.”

“Colonized substrate” as used herein refers to an inoculated substrate that has been incubated for sufficient time to allow for fungal colonization. A colonized substrate of the present disclosure can be characterized as a contiguous hyphal mass grown throughout the entirety of the volume of the growth media substrate. The colonized substrate may further contain residual nutrition that has not been consumed by the colonizing fungus. As is understood by persons of ordinary skill in the art, a colonized substrate has undergone primary myceliation, sometimes referred to by skilled artisans as having undergone a “mycelium run.” Thus, in some particular aspects, a colonized substrate consists essentially of a substrate and a colonizing fungus in a primary myceliation phase. For many fungal genera, asexual sporulation occurs as part of normal vegetative growth, and as such could occur during the colonization process. Non-limiting examples of such fungi include Pleurotus, Hericium, Morchella, Flammulina, Lentinula, Ganoderma, Grifola, Laetiporus, Polyporus, Cerioporus, Laricifomes, Fomes, and Fomitopsis. Accordingly, in some embodiments, a colonized substrate of the present disclosure may also contain asexual spores (conidia). In some aspects, a colonized substrate of the present disclosure can exclude growth progression into sexual reproduction and/or vegetative foraging. Sexual reproduction includes fruiting body formation (e.g., primordiation and differentiation) and sexual sporulation (meiotic sporulation). Vegetative foraging includes any mycelial growth away from the colonizing substrate (such as aerial growth). Thus, in some further aspects, a colonized substrate can exclude mycelium that is in a vertical expansion phase of growth. A colonized substrate can enter a mycelial vertical expansion phase during incubation in a growth environment of the present disclosure. For example, a colonized substrate can enter a mycelial vertical expansion phase upon introducing aqueous mist into the growth environment and/or depositing aqueous mist onto colonized substrate and/or any ensuing extra-particle growth. In some embodiments, the use of aqueous mist can be adjusted, for example, to desired levels and timing, to affect the topology of the growth.

“Growth environment” as used herein refers to an environment that supports the growth of mycelia, as would be readily understood by a person of ordinary skill in the art in the mycelial cultivation industry. A growth environment can contain a growth atmosphere having a gaseous environment of carbon dioxide (CO₂), oxygen (O₂) and a balance of other atmospheric gases including nitrogen (N₂) and can be further characterized as having a relative humidity. Since mushrooms are the fruiting bodies of mycelium, it should be recognized that the conditions under which mushrooms develop from mycelium (i.e., the conditions that actually trigger mycelium to produce their mushrooms (fruiting bodies)) may vary from the conditions useful to maintain mycelium in its mycelial vegetative form (such as for example, primarily, and desirably prolific aerial mycelium material growth without the production of fruiting bodies, e.g., fruiting bodies visible to the naked eye and/or fruiting bodies that comprise less than 1% of the surface area of the extra-particle mycelial growth). Essentially, the growth environment necessary to promote the extended growth of mycelium may vary from the growth environment necessary to trigger the formation of mushrooms and may also depend on the mushroom strain being grown. A growth environment of the present disclosure can be further characterized as having an atmospheric pressure as would be readily understood by a person of ordinary skill in the art in the mycelial cultivation industry.

“Aerial mycelium” as used herein refers to mycelium obtained from extra-particle aerial mycelial growth, and which is substantially free of growth matrix.

“Extra-particle mycelial growth” (EPM) as used herein refers to mycelial growth, which can be either appressed or aerial.

“Extra-particle aerial mycelial growth” as used herein refers to a distinct mycelial growth that may occurs outward (such as for example either upward, downward, or radially) from the surface of a growth matrix and which may exhibit negative gravitropism if it occurs upward. In a geometrically unrestricted scenario, extra-particle aerial mycelial growth could be described as being negatively gravitropic, positively gravitropic, or neutrally gravitropic, aerial, and radial in which growth will expand in all directions from its point source. In some embodiments, external forces, such as airflow, can be applied towards (e.g., approximately perpendicular to) the growth substrate, and in some embodiments, through the growth substrate, for example, to create downward aerial mycelium growth in the direction of gravity. Alternatively, airflow can be applied across the growth substrate in a manner parallel to the growth substrate surface.

“Extra-particle appressed mycelial growth” as used herein refers to a distinct mycelial growth that is surface-tracking (thigmotropic), is determinate in growth substantially orthogonal to the surface of a growth matrix, and is indeterminate in growth substantially parallel to the surface of the growth matrix. Extra-particle appressed mycelial growth can exhibit positive gravitropism.

“Positive gravitropism” as used herein refers to growth that preferentially occurs in the direction of gravity.

“Negative gravitropism” as used herein refers to mycelial growth that preferentially occurs in the direction away from gravity. As disclosed herein, extra-particle aerial mycelial growth can exhibit negative gravitropism. Without being bound by any particular theory, this may be attributable at least in part to the geometric restriction of the growth format, wherein an uncovered tool having a bottom and side walls or a flat planar carrier sheet either contains or supports a growth matrix. With such geometric restriction, growth will primarily occur along the unrestricted dimension(s), which in the scenario is primarily vertically (negatively gravitropic) if the tool is positioned such that its opening is facing vertically upward in orientation.

“Fruiting body” as used herein refers to a fungal stipe, pileus, gill, pore structure, or a combination thereof, and may be referred to herein as “mushroom.”

“Separation zone” as used herein refers to the zone (e.g., plane) along which the EPM is divided from the growth matrix.

“Transitional layer” as used herein refers to any extra-particle aerial mycelial growth left attached to the growth matrix after the aerial mycelium is removed. The transitional layer can be further characterized as waste excluded from the final product so as to exclude any residual substrate in the aerial mycelium. In some embodiments the transitional layer is included in the depleted growth matrix, wherein the unused mycelial growth on the transitional layer becomes part of the depleted growth matrix. In some embodiments, the transitional layer is a component of a rejuvenated growth matrix.

### DISCUSSION OF THE FIGURES

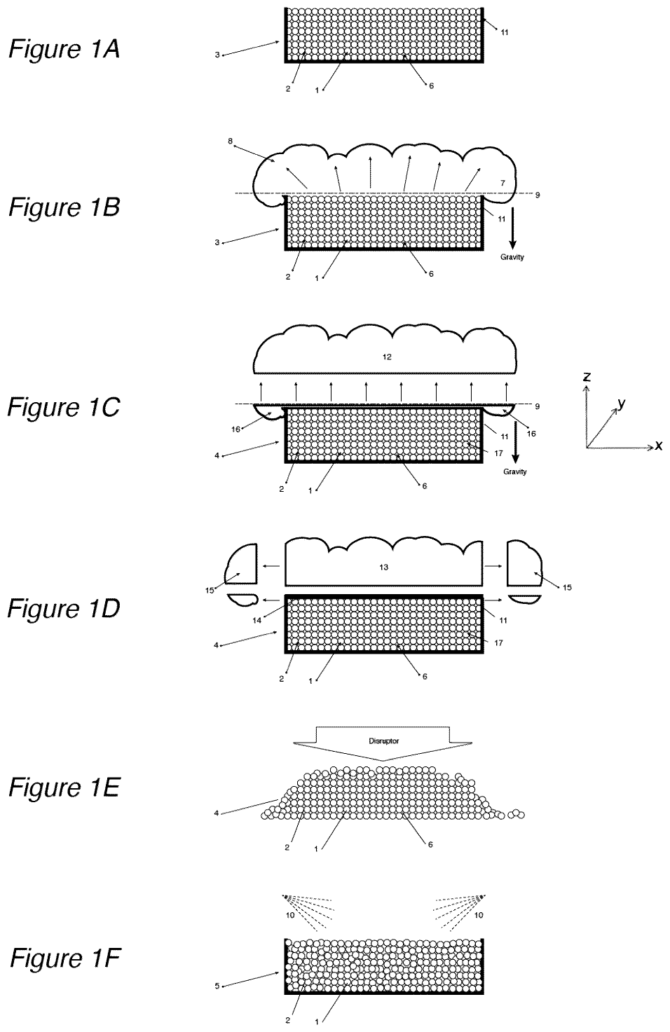

FIG. 1A illustrates an embodiment of a growth matrix 3 suitable to support negative gravitropic growth (i.e., negative gravitropism), such as extra-particle aerial mycelial growth. The growth matrix 3 is shown as circles. In some embodiments, the growth matrix 3 can be contained within a tray 11 with a bottom and side walls as shown. The growth matrix 3 can comprise growth media 2, substrate 1, and colonized (and/or precolonized) substrate 6, to support growth therefrom. In some instances, the substrate may include fresh inoculum that has not been precolonized. In some embodiments, the growth matrix 3 is implemented without tray 11 (e.g., growth matrix on another growth support structure, such as a planar support structure without side walls, such as a mycological growth web, net, screen, or planar sheet (with or without perforations)).

FIG. 1B illustrates an embodiment of negative gravitropic growth, such as extra-particle aerial mycelial growth (a type of EPM) 8 from the growth matrix 3 of FIG. 1A. For example, the growth can occur when the growth matrix 3 from FIG. 1A is incubated or otherwise processed within a growth environment under growth conditions suitable for the desired properties of the EPM 8 in FIG. 1B.

The EPM can extend upward and outward from a surface of the growth matrix to form an aerial mycelium 7. Appropriate growth conditions of the growth matrix 3 in FIG. 1A result in EPM initiating across the exposed surface of the growth matrix. Next, EPM continues to expand forming a contiguous, semi-contiguous, or discontiguous volume of extra-particle mycelial growth 8 as shown in FIG. 1B. The EPM 8 can be grown to various heights. In some embodiments, the growth is about 3-4 inches high above the exposed surface of the growth matrix 3. This can be achieved, for example, in up to two weeks of growth. It will be understood that although the EPM 8 has some amount of irregularity to its upper surface topology as shown, the drawings are not to scale, and the top surface can be relatively flat as well.

In some embodiments, the growth (such as negatively gravitropic, positively gravitropic, or radially gravitropic) can be implemented on a mycological growth web (or growing net), for example, without the tray 11 shown. The growth web can include the growth matrix and the extra-particle aerial mycelial growth (e.g., without a tray 11). The growth web can include any suitable support structure to support the growth matrix 3 and the EPM 8, such as a growing net. The growth web can be a standard size, such as a 63″W×38′L, 63″W×98′L, or any of many other growth web configurations. Other sizes can be implemented, including lengths up to 90 feet, 100 feet, or more. The growth web can comprise one or more layers of a perforated or nonperforated material, or combinations thereof, such as a plastic web, net, or film material, nylon material (e.g., nylon weave), or any other flexible, suitable material or multiple layers of material for growing EPM growth 8 from a growth matrix 3, such as for example, a nonwoven web material (e.g. a spunbond, meltblown, spunbond-meltblown laminate, or spunbond or meltblown-film laminate), or a woven web material. The growth web can extend in length from right to left in the orientation shown in FIG. 1B.

FIG. 1C illustrates an embodiment in which the EPM 8 and growth matrix 3 in FIG. 1B have been divided to form a separated aerial mycelium 12 and depleted growth matrix 4. Referring to FIG. 1B, a separation zone 9 (dot-dashed line) can be defined as a zone where the EPM 8 can be divided and detached from the growth matrix 3. As shown in FIG. 1C, upon detachment, the extra-particle aerial mycelial growth 8 from FIG. 1B has formed a separated aerial mycelium 12, and the growth matrix 3 from FIG. 1B has formed a depleted growth matrix 4.

The separation zone 9 can be positioned, and thus the growth matrix 3 and the EPM 8 can be divided, such that the depleted growth matrix 4 includes a transitional layer 14 of EPM 8 remaining upon the underlying remainder portion 17 of the depleted growth matrix 4 (See, e.g., FIG. 1D). This transitional layer is generally thin but can be of any sufficient height to prevent any of the growth matrix material 3 from remaining on the separated aerial mycelium 12 after detachment from the EPM 8 (e.g., ⅛ inch or ¼ inch). The transitional layer 14 can also allow for a cleaner, sharper detachment. The inclusion of a transitional layer 14 within the depleted growth matrix 4 can be beneficial, for example, in food applications, where the product resulting from the separated aerial mycelium 12 may not be allowed to include any significant amount of growth matrix 3. The separation zone 9 need not be linear as shown, although in some embodiments, it can form a plane extending along the dot-dashed lines shown and approximately perpendicular into the view as shown, to form a plane of separation. For embodiments that implement the tray 11, the division of the EPM 8 from the growth matrix may result in portions 16 of the EPM 8 that extend below the separation zone 9 to be divided and detached from the separated aerial mycelium 12.

FIG. 1D illustrates an embodiment of the separated aerial mycelium 12 in FIG. 1C that has been cut to form an aerial mycelium panel 13. The cut can be made transversely into the page in the orientation shown (e.g., in the y-direction). This cut would be across the width of the separated aerial mycelium 12. The portions 15 that are shown cut away from the aerial mycelium panel 13 may be, in some embodiments, part of other aerial mycelium panels 13 that are longitudinally adjacent to panel 13. For example, embodiments without a tray 11 and which implement a mycological growth web may include adjacent panels 13 rather than portions 15.

The dividing and cutting processes described with respect to FIGS. 1C and 1D, respectively, can be performed with any dividing instrument and cutting instrument, respectively, suitable to divide and/or cut mycelial growth. For example, these instruments can comprise a wire saw, a reciprocating saw (e.g., a reciprocating blade or wire), a recirculating saw (e.g., a recirculating band or wire), any of which can be configured to move in two axes—e.g., across the width of the separated aerial mycelium, and transversely, and/or a single axis “guillotine” cut (e.g., transversely), or combinations thereof. In some embodiments, a rotating cutting instrument, such as a cutting mill or “pizza cutter” configuration, can be implemented. In some embodiments, a double cake slicer blade can be implemented.

FIG. 1E illustrates an embodiment of the depleted growth matrix 4 (also referred to herein as depleted substrate) in FIGS. 1C and 1D, to which a mechanical force (e.g., a sheer force) is being applied to disrupt the depleted growth matrix. In some embodiments, the depleted growth matrix 4 is moved from the initial growth support structure (e.g., tray) to another location as shown, during the application of this mechanical force. In other embodiments, the mechanical force can be applied to the depleted growth matrix while the depleted growth matrix is on or within the initial growth structure, such as tray 11 in FIG. 1D. The mechanical force can be applied, for example, in the direction indicated by the arrow labeled “disruptor,” or in other directions. The force is applied to disrupt the depleted growth matrix 4. The disruption can increase the future growth rate within the depleted growth matrix after it is rejuvenated and used for an additional aerial mycelium growth cycle. In some embodiments, disruption can facilitate growth by increasing the surface area of mycelium available for gas exchange. In some embodiments, disruption breaks apart dense masses of mycelium which hydrophobicity hinders hydration of the depleted growth matrix. In some embodiments, disruption facilitates downstream rejuvenation, e.g., by facilitating the mixing, repacking, hydrating and/or supplementing with additive(s) of the depleted growth matrix. The mechanical force can be applied with any suitable disrupter instrument, such as a grinder, mixer, cutter, divider, blower, vibrator, perforator, lump breaker (i.e., with a breaker bar and a screen), homogenizer, bar trommel, ribbon mixer, hopper/auger, hammer mill, or any other device that can disrupt the surface (e.g., increase the surface area) and/or internal volume (e.g., increase the internal volume, and thus further increase the internal surface area) of the depleted growth matrix 4. For example, the disrupter instrument can comprise any suitable device that can aerate, grind, break, fracture, cut, perforate, fragment, or otherwise physically manipulate the depleted growth matrix 4. Such disruption can also be performed manually by production staff as they utilize their protected hands, or hand tools to disrupt the depleted growth matrix. Such disruption can improve the ability of the depleted growth matrix to be used on a subsequent EPM 8 and harvest of a subsequent aerial mycelium 7, aerial mycelium panel 13, or other product(s) such as one or more mushrooms (if desired and under appropriately specific growth conditions which preferentially foster growth of mushrooms at the cost of aerial mycelium growth). The transitional layer 14 can be retained or removed from the underlying remainder portion 17 of the depleted growth matrix 4 prior to disrupting the depleted growth matrix 4 (e.g., prior to growing a second extra-particle aerial mycelial growth or one or more mushrooms, as described further herein).

In some embodiments, the depleted growth matrix 4 can be rejuvenated to further improve growth from the depleted growth matrix 4. In some embodiments, the depleted growth matrix can be both rejuvenated and disrupted simultaneously. For example, mechanical force can be applied to the depleted growth matrix through a mixer or other disrupter instrument at the same time that the depleted growth matrix is being hydrated or otherwise being rejuvenated (i.e., with at least a partially overlapping time span). In some embodiments, the depleted growth matrix can be rejuvenated and disrupted at different times (e.g., sequentially, without an overlapping time span between the rejuvenation and disruption steps).

In some embodiments, the depleted growth matrix can be rejuvenated and disrupted by implementing a multiple-step approach which includes more than one disruption sub-step and/or more than one rejuvenation sub-step. For example, a staggered approach can be implemented whereby, e.g., the depleted growth matrix is partially disrupted, then the partial disruption sub-step is discontinued. The depleted growth matrix can be partially rejuvenated, such as by being further manipulated or supplemented (e.g., some amount of time after the partial disruption step has started). The partial rejuvenation can then be discontinued. A second partial disruption sub-step can be started (e.g., some amount of time after the first partial rejuvenation sub-step has started), and so forth, alternating between partial disruption and partial rejuvenation sub-steps until the depleted first growth matrix becomes suitable for use as a second growth matrix (that has been rejuvenated to a desired level to sustain either additional primarily, aerial mycelium material growth or the growth of mushrooms, again, under their appropriately targeted respective growth conditions).

In some embodiments, the disruption and the rejuvenation can be provided by the same instrument. For example, a mechanical disrupter instrument that can stir, mix, move, or otherwise provide mechanical force to disrupt the depleted growth matrix can include features that also provide at least one of the rejuvenating functions to the depleted growth matrix. For example, a nozzle or sprayer can be configured to contact, aerate, and otherwise disrupt the depleted growth matrix, and to also flow fluid onto, into or through the depleted growth matrix, e.g., for hydration. In some embodiments, two different fluids can be implemented to provide the two different functions. For example, an instrument can include a nozzle to direct a first fluid, such as air, at a sufficiently high pressure to disrupt (e.g., aerate) the depleted growth matrix. This disruption can be implemented with or without contacting the depleted growth matrix with the nozzle itself. The same instrument can direct a second fluid, such as water, to the depleted growth matrix for rejuvenation. The second fluid can be provided through a different nozzle or the same nozzle as the first fluid. In some embodiments, the same instrument can be implemented to provide a fluid media that provides both disruption and rejuvenation functions. For example, a nozzle can be configured to direct fluid (e.g., water) to the depleted growth matrix at a pressure sufficient to disrupt the depleted growth matrix, while also rejuvenating the growth matrix through hydration or by providing other fluid-born additive(s).

FIG. 1F illustrates an embodiment of the depleted growth matrix 4 in FIGS. 1D and 1E, which has been disrupted as described with respect to FIG. 1E, and repacked and/or redistributed in a form that can facilitate additional growth (e.g., in the tray 11 or in other forms, such as on a mycological growth web). In FIG. 1F, the depleted growth matrix 4 from FIG. 1D has been or is in the process of being rejuvenated, for example, with a rejuvenation process 10 to form a rejuvenated growth matrix 5. The depleted growth matrix 4 can be rejuvenated in many different ways that would preferentially increase its viability to support a second growth of either aerial mycelium or mushrooms thereupon. Thus, a rejuvenated growth matrix 5 can allow the depleted growth matrix (FIGS. 1D and 1E) to be reused on a subsequent EPM 8 and harvest of a subsequent aerial mycelium 7, aerial mycelium panel 13, or other product(s) such as mushrooms (either the original species or a second species) in either a second aerial mycelium growth cycle or a growth cycle (with targeted conditions) specifically calibrated to foster the growth of fruiting bodies (mushrooms). In some embodiments, the second aerial mycelium growth cycle can result in a second depleted growth matrix, which can be again rejuvenated and reused in a third aerial mycelium growth cycle, and so forth, for up to at least 2, 3, 4, or 5 aerial mycelium growth cycles, or potentially more. Alternatively, the disrupted or rejuvenated growth matrix product from repetitive prior mycelium growth cycles can be used later to grow one or more mushrooms instead of mycelium (e.g., mushrooms of the fungal organism from the original inoculation, or alternatively, a later, different fungal inoculum, and under the appropriately tailored growth conditions that are receptive to the production of mushrooms specifically, rather than extended aerial mycelium growth).

In some embodiments, the resulting growth and/or harvest can provide a subsequent, second material (e.g., a separated aerial mycelium material 12 and/or aerial mycelium panel 13) which has at least some physical and/or performance properties which are substantially the same, or different, as desired, as the first growth and/or harvest from the depleted growth matrix. In some embodiments, the resulting growth and/or harvest can provide a subsequent, second material (e.g., a separated aerial mycelium material 12 and/or aerial mycelium panel 13) which has at least some physical and/or performance properties which are substantially different as the first growth and/or harvest from the depleted growth matrix. Thus, the first and second (and later) growth cycles may be manipulated based on rejuvenation treatments to differentiate the resulting first and second (and later) growth materials from one another, such as to address different end product needs or uses. Examples of such properties or attributes and physical characteristics, which may be the same or different between the first and second (and later) growth materials include dry mass mycelium per dry mass of substrate, total wet weight mycelium yield per tool area, contiguity of aerial mycelium, morphological homogeneity, texture, elasticity, tensile strength, rigidity, density, shear strength, and compressive strength. In some embodiments, the resulting property of the second growth is at least 10%, 20%, 30%, 40%, or 50% of the same property of the first growth, under otherwise similar growth conditions. In this way, the resulting second growth and/or harvest from the rejuvenated growth matrix 5 can provide a subsequent, second material (e.g., a separated aerial mycelium 12 and/or aerial mycelium panel 13) which is substantially the same (or different, as desired) as the first growth and/or harvest from the depleted growth matrix 4, under the same or similar growth conditions. In some embodiments, the second growth can comprise a different species relative to the first growth. For example, the second growth may comprise a species that can grow under less desirable growth conditions, and/or with less desirable growth matrix, relative to the first growth. For example, the second growth may be a species that is desirable for one use (e.g., food), whereas the first growth may be desirable for another use (e.g., non-food). Alternatively, the first growth may be desirable for one type of food, whereas the second growth may be desirable for a different type of food. Alternatively, the first growth may be desirable for one type of non-food, whereas the second growth may be desirable for a different type of non-food.

Examples of rejuvenation processes can include sterilizing the depleted growth matrix 4. The sterilization can include sterilization by heat, pressure, chemicals, and/or other methods (e.g., suitable for the food industry). The sterilization can be implemented to kill any inoculum and/or any undesirable contamination for regrowth. In some embodiments, sterilization can be completed with heat and pressure by placing the depleted growth matrix in a pressure cooker. For example, the depleted growth matrix can be heated and pressurized in a pressure cooker at 15 psi for one hour. Sterilization can include many different steps, such as regrinding and repacking depleted growth matrix, mixing depleted growth matrix with fresh (i.e., non-depleted) substrate, growth media, growth matrix, and/or fungal inoculum, rehydrating the depleted growth matrix 4 (e.g., with water), rinsing the depleted growth matrix with a chemical solution (e.g., acids to mitigate hydrophobicity caused by excess chitin accumulation, excess exudates (i.e., from mycelium)), and/or supplementing the depleted growth matrix 4 with an additive(s) (e.g., charcoal). The additive(s) can include fresh fungal inoculum, fresh substrate, fresh growth media, fresh growth matrix, charcoal, a nutrient source, and/or other additive(s) to further improve the viability of the rejuvenated growth matrix. For example, the additive(s) can include any substance that was in the initial growth matrix prior to it being processed to form the depleted growth matrix. In some implementations, additive(s) can include pH modifiers, including sodium carbonate, activated charcoal and/or wood ash. In some implementations, additive(s) can include water combined with a surfactant (e.g., lecithin and/or Tween™) to reduce hyphal hydrophobicity and to facilitate mycelium hydration, including with tap water or water with conductivity up to 800 uS/cm or filtered water (e.g., conductivity less than 50 uS/cm) and/or enriched mineral solutions (e.g., conductivity greater than 2000 uS/cm). In some implementations, additive(s) can include a carbohydrate, including soy flour, soy bran, wheat bran, starch, wheat flour, soy hulls, corn starch, and/or cookie meal. In some implementations, additive(s) can include a carbon source, including glucose, maltodextrin, xylose (C5 sugars) and/or corn sugar. In some implementations, additive(s) can include mineral compounds, including manganese, magnesium, zinc, copper, iron, calcium and/or phosphorous. In some implementations, additive(s) can include plant seeds or seed meal to provide supplemental fatty acids, starch, and minerals. In some embodiments, the rejuvenation of the depleted growth matrix can be biological in nature, for example, with a biological additive(s). For example, fermentation, bacteria, biological organisms, and/or other biological features can be implemented to rejuvenate a growth matrix. For example, biological rejuvenation may be implemented which can reduce chitin in the depleted growth matrix. In some aspects, the rejuvenation process can involve applying a covering layer to the depleted substrate. A covering layer is a layer of organic and/or inorganic material placed on top of and/or below the depleted growth matrix. The covering layer can comprise non-depleted substrate, growth media and/or growth matrix. A covering layer can comprise, for example, vermiculite, peat moss, coconut coir, or any material that can be used as an uninoculated substrate, or a combination of such materials. In some embodiments, the covering layer can comprise fresh inoculum, such as fungal inoculum. For example, the covering layer can comprise a fresh growth matrix layer on top or underneath the depleted growth matrix. In some embodiments, the covering layer, or other rejuvenating processes herein, can be implemented without including a disruption step, such that depleted growth matrix is merely exposed to a rejuvenation step and then allowed to grow either additional aerial mycelia or mushrooms.

In some embodiments, one or more features can be implemented instead of or in addition to a rejuvenation and/or disruption step, to facilitate a multi-cycle growth process. For example, the growth matrix can include a detergent or other chemical that will hold hydration within the growth matrix for more than one aerial mycelium growth cycle. In some embodiments, the growth matrix can include components that provide time-delayed chemicals that facilitate a second aerial mycelium growth cycle after a first growth product is harvested. For example, gel caps can be introduced into a growth matrix that slowly release or provide a delayed release of water and/or nutrients through more than one aerial mycelium growth cycle.

Upon harvesting of the separated aerial mycelium 12 (FIG. 1C) or aerial mycelium panel (FIG. 1D), and completion of the disruption (FIG. 1E) and rejuvenation (FIG. 1F), the steps illustrated in FIGS. 1A-1F can be repeated on the rejuvenated growth matrix 5 one or more times, to provide two or more subsequent harvests with at least some of the same growth matrix from the first harvest.

In some embodiments, the disrupting process (e.g., described with respect to FIG. 1E), and/or the rejuvenating process (e.g., described with respect to FIG. 1F) can be performed on the depleted growth matrix “in place” without removing the depleted growth matrix from its underlying support structure, such as a tray or a mycological growth web. In such embodiments, the second growth and subsequent processing steps can occur on the same support structure or same growth/harvesting equipment to simplify production.

In some embodiments, the depleted growth matrix can be removed from its underlying support structure, such as the tray or mycological growth web. In some embodiments, the depleted growth matrix can be stored, for example, for up to 1, 2, 3, or 4 days, up to 1, 2, 3, or 4 weeks, up to 1 month, or up to 2 months prior to using it for a further aerial mycelium or mushroom growth cycle. In some embodiments, the depleted growth matrix can be stored, for example, in a fluidized bed and/or a temperature-controlled device prior to using it for a further aerial mycelium or mushroom growth cycle. In other embodiments, the depleted growth matrix can be processed immediately after removal from its underlying support structure for use in a further aerial mycelium growth cycle (or alternatively a mushroom growth cycle).

In some embodiments, the depleted growth matrix can be processed as described herein to become rejuvenated growth matrix. In some embodiments, rejuvenated growth matrix can be immediately placed onto a new support structure and into a growth environment to begin a further aerial mycelium growth cycle (or alternatively a mushroom growth cycle, again, under specifically targeted growth conditions catering to the differing needs of either aerial mycelium or mushrooms). In other embodiments, the rejuvenated growth matrix can be stored, for example, for up to 1, 2, 3, or 4 days, up to 1, 2, 3, or 5 weeks, up to 1 month, or up to 2 months prior to using it for a further aerial mycelium growth cycle (or alternatively a mushroom growth cycle). In some embodiments, the rejuvenated growth matrix can be stored, for example, in a fluidized bed and/or a temperature-controlled device prior to using it for a further aerial mycelium growth cycle (or alternatively a mushroom growth cycle).

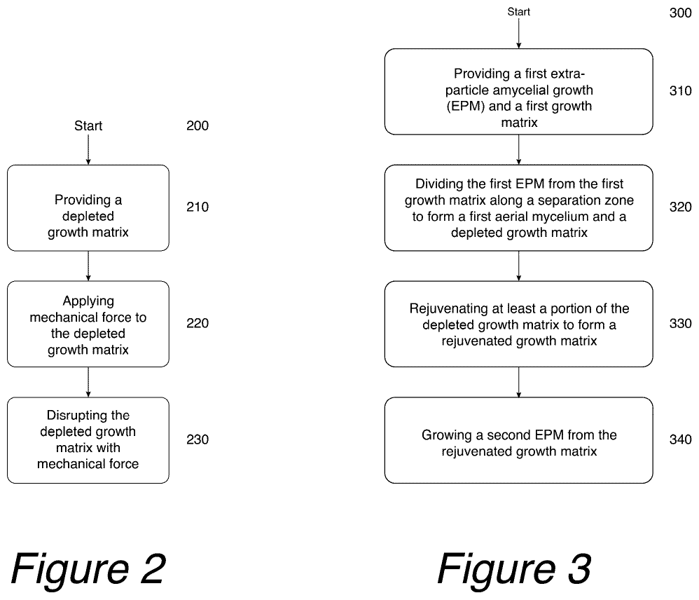

FIG. 2 illustrates a method 200 of growing an aerial mycelium material. The method 200 can include a step 210 of providing a depleted growth matrix, such as that described with respect to FIG. 1C. The method 200 can include a step 220 of applying a mechanical force to the depleted growth matrix, and a step 230 of disrupting the depleted growth matrix with a mechanical force, such as that described with respect to FIG. 1E. In some implementations of method 200, disrupting can include increasing the surface area of the depleted growth matrix thereby increasing gas permeability through the growth media, and consequently increasing metabolic efficiency. In some embodiments, disruption can facilitate rehydration by dismantling dense masses of hydrophobic mycelium. In some implementations, disruption may be accompanied by nutritive modification, pH modification, increased hydration, and/or increased oxygenation. In some implementations of method 200, disrupting can include varying degrees of shear force. Examples of disruption include at least one of grinding, breaking, gouging, tilling, fracturing, cutting, perforating, fragmenting, and physically manipulating the depleted growth matrix. Disruption can include direct-contact processes or contactless processes, such as vibration, sonic exposure, or others. In one embodiment of the method, the mechanical force causing a disruption can entail the matrix breaking or cracking under its own weight (such as if it were in the form of a solid planar sheet or brick that fractures) as it traverses a void or gap beneath it, or is forced to move across an inclined surface during a production process, and with gravity acting upon it, causing it to snap apart at various locations along its length under its own weight. In an alternative embodiment, following such disruption, the depleted growth matrix may be used (e.g., without further supplementation) as the basis of a second extra-particle aerial mycelial growth cycle or alternatively as the basis of a fruiting body (e.g., mushroom) growth cycle (again under specifically targeted and appropriate conditions that encourage the growth of the different end products). In a further alternative embodiment, following such disruption, the depleted growth matrix may be rejuvenated as hereinafter described and then used as the basis of a second extra-particle aerial mycelial growth cycle or alternatively as the basis of a fruiting body (e.g., mushroom) growth cycle. In the instances in which the mere depleted growth matrix or alternatively rejuvenated growth matrix are used as the basis of a fruiting body (e.g., mushroom) growth cycle, such depleted or rejuvenated growth matrix may be packed into manageable-sized containers, such as tools or bags, and held under mushroom-favoring growth conditions to promote mushroom growth as opposed to aerial mycelium growth. Such manageable-sized containers may also be temporarily stored under conditions that would discourage growth without spoilage of the fungal organism before initiating growth under mushroom-favoring conditions.

In some implementations, method 200 can include rejuvenating at least a portion of the depleted growth matrix to form a rejuvenated growth matrix and growing extra-particle aerial mycelial growth from the rejuvenated growth matrix. In some such implementations, the method 200 can further include detaching the extra-particle aerial mycelial growth from the rejuvenated growth matrix to form a separated aerial mycelium from the extra-particle aerial mycelial growth, wherein the extra-particle aerial mycelium and the separated aerial mycelium do not comprise a fruiting body. In some such implementations, rejuvenating can include at least one of: sterilizing the depleted growth matrix, rehydrating the depleted growth matrix with water of conductivity ranging from 50-2000 uS/cm and/or varying mineral content, and/or supplementing the depleted growth matrix with an additive(s). In some such implementations of method 200, the additive(s) can comprise at least one of fresh fungal inoculum, fresh substrate, and a nutrient source. The method 200 can further include one or more of the steps described herein with respect to FIGS. 3, 4 and 5 and the embodiments shown in FIGS. 1A-1F, except where inconsistent with the disclosure herein of method 200. In some aspects, the rejuvenating step can be performed before, concurrently with, or following the disrupting step.

FIG. 3 illustrates a method 300 of growing an aerial mycelium material. The method 300 can include a step 310 of providing a first extra-particle aerial mycelial growth and a first growth matrix, such as that described with respect to FIG. 1B. In step 310, the first extra-particle aerial mycelial growth can extend from a surface of the first growth matrix.

The method 300 can include a step 320 of dividing the first extra-particle aerial mycelial growth from the first growth matrix along a separation zone to form a first aerial mycelium and a depleted growth matrix such as that described with respect to FIG. 1C. In some such implementations of method 300, dividing can include dividing the first extra-particle aerial mycelial growth from the first growth matrix along the separation zone such that the depleted growth matrix includes a transitional layer of unused extra-particle aerial mycelial growth on the uppermost surface of an underlying remainder portion of the depleted growth matrix. Some implementations of method 300 can include dividing the second extra-particle aerial mycelial growth and the rejuvenated growth matrix along a second separation zone to form a second separated aerial mycelium and a second depleted growth matrix and cutting the second separated aerial mycelium in a transverse direction and across a width of the separated aerial mycelium to form an aerial mycelium panel.

The method 300 can include a step 330 of rejuvenating at least a portion of the depleted growth matrix to form a rejuvenated growth matrix such as that described with respect to FIG. 1F. In some implementations of method 300, rejuvenating includes adjusting the moisture content of the depleted growth matrix to between 40% and 75%, with a water activity (aW) greater than 0.95. In some such implementations, moisture content prior to myceliation can range from about 60% to about 75% (e.g., about 65%), which can be reduced to 50% after the first aerial mycelium growth. In some implementations of method 300, rejuvenating comprises rehydrating, wherein rehydrating comprises gouging the substrate surface. Gouging entails the process of creating holes from 1 to 3 cm in diameter or other suitable size in the depleted growth matrix using, e.g., a pipette tip or any suitable gouging or piercing instrument.

The method 300 can include a step 340 of growing a second extra-particle aerial mycelial growth from the rejuvenated growth matrix. Step 340 can be implemented, for example, by implementing the rejuvenated growth matrix in FIG. 1F, and incubating it as described with respect to FIG. 1B. In some such implementations, method 300 can include removing the transitional layer from the underlying remainder portion of the depleted growth matrix prior to disrupting the depleted growth matrix and growing the second extra-particle aerial mycelial growth. In some such implementations, the first extra-particle aerial mycelial growth, the first separated aerial mycelium, and the second extra-particle aerial mycelial growth do not comprise a fruiting body.

The method 300 can further include one or more of the steps herein described with respect to FIGS. 2, 4 and 5, and the embodiments shown in FIGS. 1A-1F, except where inconsistent with the disclosure herein of method 300.

In a further alternative embodiment of the method of FIG. 3, the step 340 of growing a second EPM from the rejuvenated growth matrix may be controlled to lead to the production of fruiting bodies (e.g., mushrooms). Conditions amenable to the growth of mushrooms (and which are known in the field of mushroom harvesting) rather than abundant aerial mycelium growth may be created to foster such a growth of mushrooms.

As an alternative in which the rejuvenated growth matrix can be controlled to lead to the production of fruiting bodies (e.g., mushrooms) by selectively controlled growth conditions designed to foster preferred mushroom growth rather than enhanced mycelium growth, the depleted growth matrix may be ground or otherwise disrupted as described in earlier embodiments with respect to depleted growth matrix. The depleted growth matrix can optionally be supplemented with additional nutrients as previously described with respect to depleted growth matrix. The depleted growth matrix (either supplemented or not) may then be placed into containers (such as for example in 6-20 lb bags, such bags optionally having an array of holes to induce pinning) or in a bulk tote (such as a container capable of holding between 800 and 1000 lbs). The containers or bulk totes may be used solely for transport to a secondary bagging or packaging step or for transport under appropriate transport and/or storage conditions to a location in which a targeted mushroom crop is to be grown, such as under growth conditions which preferably foster the growth of mushrooms rather than aerial mycelium proliferation. As an example, the incubation of a mushroom crop may span from 9 to 28 days, with humidity of greater than 60% and an incubation temperature of between 72-76° F.

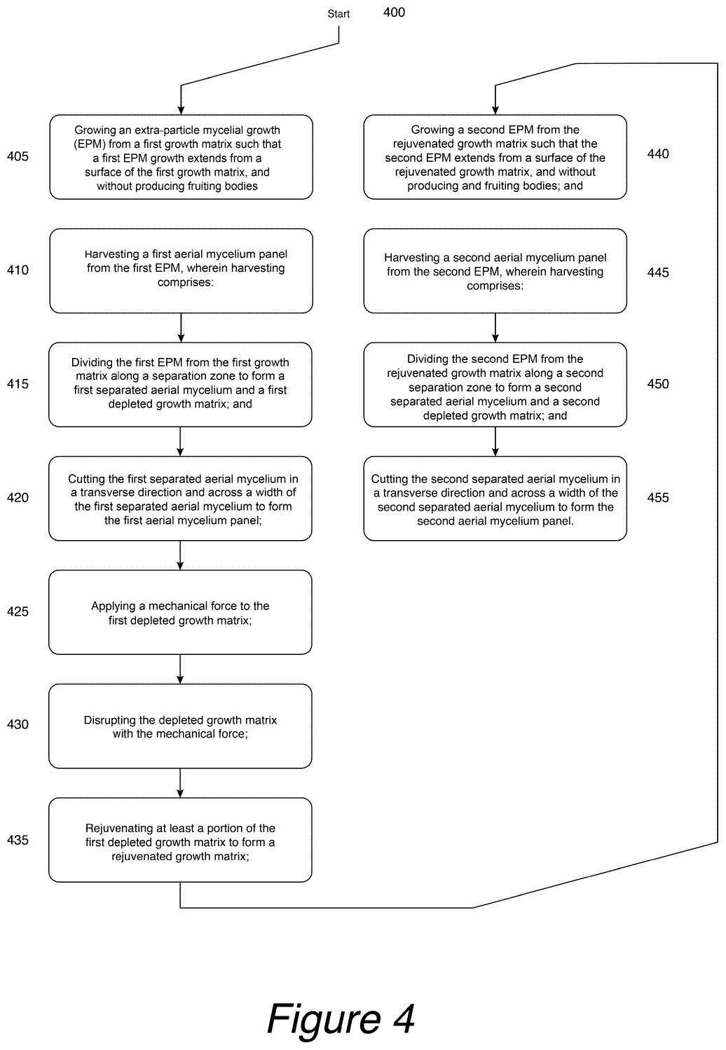

FIG. 4 illustrates a method 400 of growing aerial mycelium panels. The method 400 can include a step 405 of growing a first extra-particle aerial mycelial growth from a first growth matrix. The first extra-particle aerial mycelial growth can extend from a surface of the first growth matrix, and without producing any fruiting bodies, such as that described with respect to FIG. 1B.

The method 400 can include a step 410 of harvesting a first aerial mycelium panel from the first extra-particle aerial mycelial growth. Harvesting can comprise a step 415 of dividing the first extra-particle aerial mycelial growth from the first growth matrix along a separation zone to form a first separated aerial mycelium and a first depleted growth matrix. Harvesting can further comprise a step 420 of cutting the first separated aerial mycelium in a transverse direction and across a width of the first separated aerial mycelium to form the first aerial mycelium panel, such as that described with respect to FIGS. 3C and 3D.

The method 400 can include a step 425 of applying a mechanical force to the first depleted growth matrix. The method 400 can further include a step 430 of disrupting the depleted growth matrix with the mechanical force, such as that described with respect to FIG. 1E.

The method 400 can include a step 435 of rejuvenating at least a portion of the first depleted growth matrix to form a rejuvenated growth matrix, such as described with respect to FIG. 1F.

The method 400 can include a step 440 of growing a second extra-particle aerial mycelial growth from the rejuvenated growth matrix. The second extra-particle aerial mycelial growth can extend from a surface of the rejuvenated growth matrix, and without producing any fruiting bodies. Step 440 can include similar process parameters as step 405. Step 440 can be implemented, for example, by implementing the rejuvenated growth matrix in FIG. 1F and incubating it as described with respect to FIG. 1B.

The method 400 can include a step 445 of harvesting a second aerial mycelium panel from the second extra-particle aerial mycelial growth. Harvesting can comprise a step 450 of dividing the second extra-particle aerial mycelial growth from the second growth matrix along a separation zone to form a second separated aerial mycelium and a second depleted growth matrix. The harvesting step 450 can further comprise a step 455 of cutting the second separated aerial mycelium in a transverse direction and across a width of the second separated aerial mycelium to form the second aerial mycelium panel, such as that described with respect to FIG. 3C and FIG. 3D.

In some implementations of method 400, the first and/or the second depleted growth matrix can comprise a transitional layer of unused extra-particle aerial mycelial growth and an underlying remainder portion of the depleted growth matrix, and method 400 can further comprise removing the transitional layer from the underlying remainder portion. As noted previously, the transitional layer may be retained during any disrupting step or removed prior to any disrupting step.

In some implementations of method 400, the first separated aerial mycelium and the second separated aerial mycelium can be substantially the same with respect to one or more of the following physical characteristics: dry mass mycelium per dry mass of substrate, total wet weight mycelium yield per tool area, contiguity of aerial mycelium, morphological homogeneity, texture, elasticity, tensile strength, rigidity, density, shear strength, and compressive strength. In some implementations of method 400, the first and the second separated aerial mycelium can be substantially the same with respect to one, two, three or more, or all, of the physical characteristics described herein.

In some implementations of method 400, the first separated aerial mycelium and the second separated aerial mycelium can be substantially different with respect to one or more of the following physical characteristics: dry mass mycelium per dry mass of substrate, total wet weight mycelium yield per tool area, contiguity of aerial mycelium, morphological homogeneity, texture, elasticity, tensile strength, rigidity, density, shear strength, and compressive strength. In some implementations of method 400, the first and the second separated aerial mycelium can be substantially different with respect to one, two, three or more, or all, of the physical characteristics described herein.

In some implementations, the first separated aerial mycelium, the second separated aerial mycelium, the first aerial mycelium panel, and/or the second aerial mycelium panel do not comprise a fruiting body.

The method 400 can further include one or more of the steps described herein with respect to FIGS. 2, 3, and 5 and the embodiments shown in FIGS. 1A-1F, except where inconsistent with the disclosure herein of method 400.

It will be understood that the processes, process steps, apparatus and components described above and illustrated in FIGS. 1A-1F and FIGS. 2-5 are not mutually exclusive, and each can be implemented separately, for example, with other processes, process steps, apparatuses and components, or in combination with each other, or with other processes, process steps, apparatuses and components.

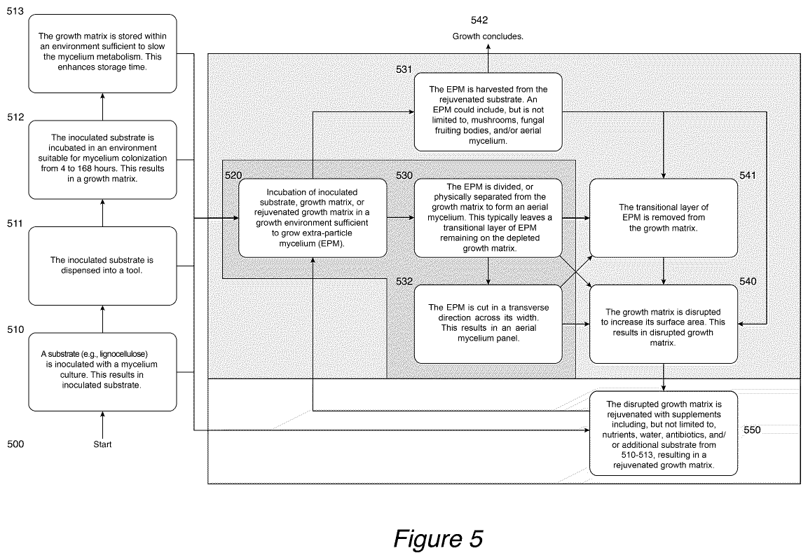

FIG. 5 illustrates a flow diagram of an embodiment of a method 500 for growing aerial mycelium material, such as aerial mycelium and aerial mycelium panels, and others. Method 500 can include many steps that are similar to those described with respect to FIGS. 2, 3, and 4, but is shown with additional preceding steps and visual details to better understand additional process flows, options and logic. The method 500 can include step 510 of inoculating a substrate (e.g., a lignocellulose material), step 511 of dispensing inoculated substrate into or onto a tool (e.g., tray or web), step 512 of incubating inoculated substrate in an environment suitable for mycelium colonization (e.g., for four to 168 hours) to produce a growth matrix, and step 513 of storing the growth matrix in an environment sufficient to slow mycelium metabolism to enhance storage time.

Any combination of steps 510-513 can flow to a step 520 of incubating either an inoculated substrate, a growth matrix or a rejuvenated growth matrix in a growth environment, which is then used to produce an EPM similar to that described with respect to FIGS. 2, 3 (e.g., step 310) and/or 4 (e.g., step 405). The method 500 can further include a step 530 of dividing the EPM to produce an aerial mycelium and a transitional layer, similar to that described with respect to FIGS. 2, 3 (e.g., step 320) and/or 4 (e.g., step 415). The method 500 can further include a step 532 of cutting the EPM, which results in an aerial mycelium panel, similar to that described with respect to FIGS. 2, 3, and/or 4 (e.g., step 420).

The transitional layer optionally can be removed in a step 541, or the transitional layer optionally can be included in a step 540 of disrupting the growth matrix (e.g., to increase its surface area), similar to that described with respect to FIGS. 2 (e.g., step 230), 3 and/or 4 (e.g., step 430).

The method 500 can include a step 550 of rejuvenating the substrate with supplements including, but not limited to, nutrients, water, antibiotics, and additional substrate, such as fresh substrate or that produced from steps 510, 511, 512, and 513. The method 500 can further include incubating the rejuvenated substrate similar to that described with respect to step 520 to produce another EPM. The EPM can be further harvested in a step 531 wherein the EPM can include, but is not limited to, aerial mycelia (primarily or solely), mushrooms, and fungal fruiting bodies. In an alternative embodiment, the EPM can include only aerial mycelia. In yet a further alternative embodiment, the EPM can include aerial mycelia of a majority species that differ from the majority species of an earlier EPM harvest prior to rejuvenation of the substrate, in that an earlier EPM harvest from the original substrate can have a majority of aerial mycelia from a particular fungal species, whereas a second or rejuvenated EPM harvest from the rejuvenated substrate can have a majority of aerial mycelia from a second fungal species.

In yet still a further alternative embodiment, either the disrupted depleted growth matrix or the rejuvenated (and formerly depleted) growth matrix can then be used to grow a product comprising primarily or entirely fruiting bodies (e.g., mushrooms), rather than aerial mycelium. In such an embodiment, the previously depleted growth matrix may demonstrate additional practical uses aside from growth of a primarily aerial mycelium material.

The method 500 can further include one or more of the steps described herein with respect to FIGS. 2, 3 and 4 and the embodiments shown in FIGS. 1A-1F, except where inconsistent with the disclosure herein of method 500.

In yet still further alternative embodiments, methods for generating aerial mycelial growth (without fruiting bodies (e.g., mushrooms)) can include the steps of providing substrate remaining from a traditional harvest of mushrooms (a type of depleted growth matrix hereinafter referred to as “remainder substrate”), which has been depleted of nutrients and/or substrate over the normal course of growth of the mushrooms (in which aerial mycelial growth was not the focus, and the growth conditions did not specifically promote such growth (and in fact discouraged such growth), and either: (1) then adding the remainder substrate (in one embodiment disrupted prior to addition) to supplement existing depleted growth matrix from aerial mycelium growth, and exposing both the remainder substrate and depleted growth matrix to conditions that would then preferably facilitate the growth of aerial mycelium rather than fruiting bodies (e.g., mushrooms) (e.g., without the occurrence of fruiting bodies), or (2) utilizing the remainder substrate as the sole basis for growing aerial mycelium under conditions (in one embodiment, following disruption of the remainder substrate) that would preferentially facilitate the growth of aerial mycelium rather than fruiting bodies (e.g., mushrooms).

For instance, in such embodiments, the remainder substrate from a mushroom harvesting operation can be ground or otherwise disrupted as described in earlier embodiments with respect to depleted growth matrix. The remainder substrate can optionally be supplemented with additional nutrients as previously described with respect to depleted growth matrix. The remainder substrate (either supplemented or not) may then be placed into containers (such as for example in 6-20 lb bags, such bags optionally having an array of holes to induce pinning) or in a bulk tote (such as a container capable of holding between 800 and 1000 lbs). The containers or bulk totes may be used solely for transport to a secondary bagging or packaging step or for transport under appropriate transport and/or storage conditions to a location in which aerial mycelium is later to be grown (under conditions specifically targeted to promote the preferential growth of aerial mycelium rather than mushrooms or fruiting bodies).

Textiles or Other Non-Food Implementations

In some aspects, the present disclosure provides for an aerial mycelium, and for methods of making an aerial mycelium, wherein the aerial mycelium is a growth product of a fungus. In some embodiments, the fungus is a species of the genus Agrocybe, Albatrellus, Armillaria, Agaricus, Bondarzewia, Cantharellus, Cerioporus, Climacodon, Cordyceps, Fistulina, Flammulina, Fomes, Fomitopsis, Fusarium, Grifola, Hericium, Hydnum, Hypomyces, Hypsizygus, Ischnoderma, Laetiporus, Laricifomes, Lentinula, Lentinus, Lepista, Meripilus, Morchella, Ophiocordyceps, Panellus, Piptoporus, Pleurotus, Polyporus, Pycnoporellus, Rhizopus, Schizophyllum, Stropharia, Tuber, Tyromyces, Wolfiporia, Ceriporiopsis, Chlorociboria, Daedalea, Daedaleopsis, Daldinia, Ganoderma, Hypoxylon, Inonotus, Lenzites, Omphalotus, Oxyporus, Phanerochaete, Phellinus, Polyporellus, Porodaedalea, Pycnoporus, Scytalidium, Stereum, Trametes or Xylaria. In some further embodiments, the fungus is a species of the genus Bondarzewia, Ceriporiopsis, Daedalea, Daedaleopsis, Fomitopsis, Ganoderma, Inonotus, Lenzites, Omphalotus, Oxyporus, Phellinus, Polyporellus, Polyporus, Porodaedalea, Pycnoporus, Stereum, Trametes or Xylaria. In some more particular embodiments, the fungus is selected from the group consisting of Bondarzewia berkeleyi, Daedalea quercina, Daedaleopsis spp., Daedaleopsis confragosa, Daedaleopsis septentrionalis, Fomitopsis spp., Fomitopsis cajanderi, Fomitopsis pinicola, Ganoderma spp., Ganoderma amboinense, Ganoderma applanatum, Ganoderma atrum, Ganoderma australe, Ganoderma brownii, Ganoderma capense, Ganoderma carnosum, Ganoderma cochlear, Ganoderma colossus, Ganoderma curtisii, Ganoderma donkii, Ganoderma formosanum, Ganoderma gibbosum, Ganoderma hainanense, Ganoderma hoehnelianum Ganoderma japonicum, Ganoderma lingzhi, Ganoderma lobatum, Ganoderma lucidum, Ganoderma multipileum, Ganoderma oregonense, Ganoderma pfeifferi, Ganoderma resinaceum, Ganoderma sessile, Ganoderma sichuanense, Ganoderma sinense, Ganoderma tropicum, Ganoderma tsugae, Ganoderma tuberculosum, Ganoderma weberianum, Inonotus spp., Inonotus obliquus, Inonotus hispidus, Inonotus dryadeus, Inonotus tomentosus, Lenzites betulina, Phellinus spp., Phellinus igniarius, Phellinus gilvus, Polyporus spp., Polyporus squamosus, Polyporus badius, Polyporus umbellatus, Polyporus squamosus, Polyporus tuberaster, Polyporus arcularius, Polyporus alveolaris, Polyporus radicatus, Porodaedalea pini, Pycnoporus spp., Pycnoporus spp., Pycnoporus sanguineus, Pycnoporus cinnabarinus, Stereum spp., Stereum ostrea, Stereum hirsutum, Trametes spp., Trametes versicolor, Trametes elegans, Trametes suaveolens, Trametes hirsuta, Trametes gibbosa, Trametes ochracea, Trametes villosa, Trametes cubensis and Trametes pubescens. In some other embodiments, the fungus is a pigment-producing fungus of a genus selected from the group consisting of Chlorociboria, Daldinia, Hypoxylon, Phanerochaete and Scytalidium. In yet some other embodiments, the fungus is a species of the genus Ganoderma. In some further embodiments, the fungus is Ganoderma spp., Ganoderma amboinense, Ganoderma applanatum, Ganoderma atrum, Ganoderma australe, Ganoderma brownii, Ganoderma capense, Ganoderma carnosum, Ganoderma cochlear, Ganoderma colossus, Ganoderma curtisii, Ganoderma donkii, Ganoderma formosanum, Ganoderma gibbosum, Ganoderma hainanense, Ganoderma hoehnelianum Ganoderma japonicum, Ganoderma lingzhi, Ganoderma lobatum, Ganoderma lucidum, Ganoderma multipileum, Ganoderma oregonense, Ganoderma pfeifferi, Ganoderma resinaceum, Ganoderma sessile, Ganoderma sichuanense, Ganoderma sinense, Ganoderma tropicum, Ganoderma tsugae, Ganoderma tuberculosum or Ganoderma weberianum. In some embodiments, the fungus is Ganoderma sessile.

Food Implementations

In some aspects, the present disclosure provides for an aerial mycelium, and for methods of generating an aerial mycelium, wherein the aerial mycelium is a growth product of a fungus. In some embodiments, the fungus is a species of the genus Agrocybe, Albatrellus, Armillaria, Agaricus, Bondarzewia, Cantharellus, Cerioporus, Climacodon, Cordyceps, Fistulina, Flammulina, Fomes, Fomitopsis, Fusarium, Grifola, Hericium, Hydnum, Hypomyces, Hypsizygus, Ischnoderma, Laetiporus, Laricifomes, Lentinula, Lentinus, Lepista, Meripilus, Morchella, Ophiocordyceps, Panellus, Piptoporus, Pleurotus, Polyporus, Rhizopus, Schizophyllum, Stropharia, Tuber, Tyromyces or Wolfiporia. In some further embodiments, the fungus is a species of the genus Pleurotus. In some more particular embodiments, the fungus is Pleurotus albidus, Pleurotus citrinopileatus, Pleurotus columbinus, Pleurotus cornucopiae, Pleurotus dryinus, Pleurotus djamor, Pleurotus eryngii, Pleurotus floridanus, Pleurotus nebrodensis, Pleurotus ostreatus, Pleurotus populinus, Pleurotus pulmonarius, Pleurotus sajor-caju, Pleurotus salmoneo-stramineus, Pleurotus salmonicolor or Pleurotus tuber-regium. In some embodiments, the fungus is Pleurotus ostreatus.

Experimental Support

The following provides example compositions and cultivation methods for producing an aerial mycelium. The examples are not intended to limit the embodiments described herein but are intended to illustrate how a growth media composition and environmental conditions can be used to produce an aerial mycelium with certain material properties. A summary of the experimental conditions and results are described in Tables I and II, provided below.

TABLE I
Experimental Support Summary for Experiment Nos. 0-4

| | | | Experiment |
|---|---|---|---|
| Status | Treatment | Outcome | No. |
| Resulted | Sterilizing, reinoculating and | 35% wet mass yield as | 0 |
| in Aerial | adding fresh nutrition to depleted | compared to concurrently grown | |
| Mycelial | substrate | control panel | |
| Tissue | Regrinding, rehydrating and | 8.7-21.5% wet mass yield as | 1, 3, 4 |
| | repacking depleted substrate | compared to concurrently grown | |
| | | control panel | |
| | Regrinding, rehydrating, | 34.5-37% wet mass yield as | 3 |
| | reinoculating and repacking | compared to concurrently grown | |
| | depleted substrate | control panel or parent run | |
| | 1:1 mixing of reground depleted | 15.6-20.5% wet mass yield as | 4 |
| | substrate with fresh, uninoculated | compared to concurrently grown | |
| | substrate | control panel or parent run | |
| | Adding charcoal powder to | No significant yield difference | 4 |
| | reduce waste buildup | as compared to concurrently | |
| | | grown control panel | |
| | Gouging substrate surface to | 9.6-10.2% wet mass yield as | 3 |
| | rehydrate it | compared to concurrently grown | |
| | | control panel or parent run | |
| Minimal/no | Flipping depleted substrate brick | Produced primordium when it | 4 |
| Aerial | over in tool | grew (not aerial mycelium) | |
| Mycelia | Dunking depleted substrate in | Did not hydrate as quickly as | 2 |
| | water to rehydrate it | desired (+2.5-5% in 24 hours) | |
| | Extracting panel and reinserting | Did not elicit aerial mycelia | 1 |
| | depleted substrate with | | |
| | transitional layer as is in tool into | | |
| | growth chamber | | |
| | Removing transitional layer and | Did not elicit aerial mycelia | 3 |
| | leaving depleted substrate in tool | | |
| | undisturbed | | |
| | Remove top ¼″ depth layer of | Did not elicit aerial mycelia | 4 |
| | substrate | | |

Experiment 0

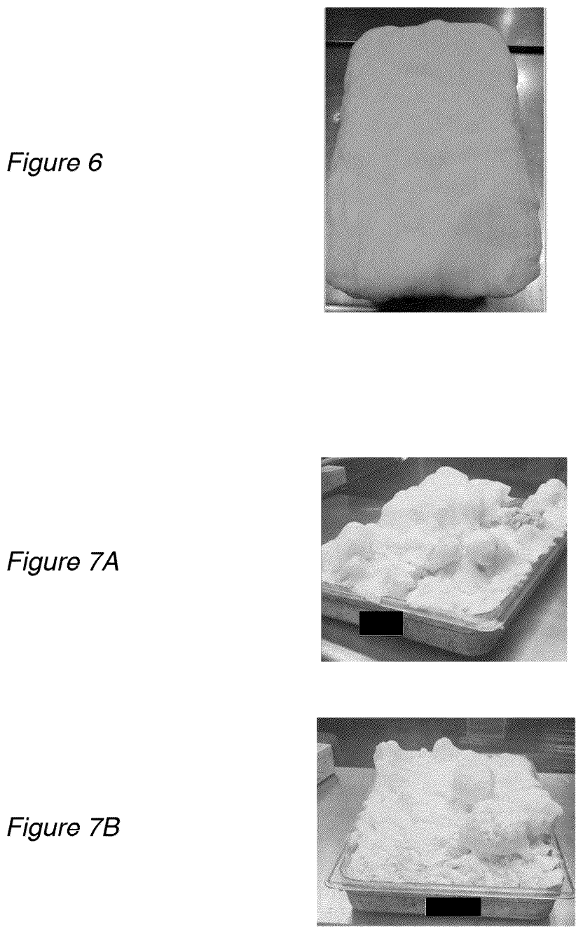

In some embodiments, depleted substrate can be sterilized and nutrition (e.g., soy flour) can be added at inoculation. In one example, depleted maple flake substrate was sterilized and soy flour with fungal inoculum was added to the depleted substrate. These conditions produced an aerial mycelium panel of 173 g, which was 35% by wet mass as compared to a concurrently grown panel grown from fresh maple flakes (i.e., a control panel). FIG. 6 shows an embodiment of a panel grown from fresh maple flakes after addition of soy flour with fungal inoculation.

Experiment 1

In a further example, depleted substrate can be removed after panels are harvested, for example with the use of an extraction saw.

In a first condition, one tool full of substrate was left complete with the transitional layer, except for 6 g of substrate removed for moisture readings, while the rest of the surface was left unperturbed. The average substrate moisture reading in this condition was 51.3%. This tool was lidded and left in a growth chamber for incubation. At extraction 13 days later, this tool did not have any measurable aerial mycelial growth.

In a second condition, the substrate removed from harvest was manually reground (i.e., removed from a tool and broken up manually, including the transitional layer) and repacked into a tool on the same day of harvest (day 13), except for 25 g removed for bacterial testing and 6 g removed for moisture readings. On the day of extraction (i.e., when the first aerial mycelium growth run was removed), the depleted substrate in this tool had an average moisture content of 56.6%. The next day, the depleted substrate in this tool was removed from the tool and rehydrated to a goal moisture content of 63%. The depleted substrate was manually mixed with water and repacked into the same tool, except for 6 g removed for moisture readings, which was 60.68%. The tool was lidded and left in a growth chamber for incubation. Thirteen days later at the second extraction, the tool weighed 115 g, which was 13.7% by wet mass as compared to the parent run (i.e., compared to the panel previously grown from the same substrate before becoming depleted and rehydrated substrate) and 16.0% by wet mass as compared to a control panel (i.e., a panel grown from fresh substrate).

Experiment 2

In another example, substrate was submerged in the amount of water that it would take to hydrate it to 63% (i.e., the substrate would have to absorb that full amount of water to reach 63% moisture content). The substrate was hydrated over 24 hours to allow sufficient time for absorption while minimizing opportunity for contamination growth. The moisture content readings provided below are not averages but individual measurements.

A first tool (“Tray 1”), which started at 49.02% moisture content and was submerged in half of the amount of water that it would take to hydrate it to 63%, ended at 53.74% moisture content.

A second tool (“Tray 2”), which started at 46.7% moisture content and was submerged in the amount of water that it would take to hydrate it to 63%, ended at 48.4% moisture content.

A third tool (“Tray 3”) was not exposed to water and acted as the control condition. Tray 3 started at 47.15% moisture content and ended at 44.68% moisture content, with a net loss of 2.47% moisture content.

Experiment 3

In an additional embodiment, depleted substrate can be used to elicit further mycelial growth. All treatments were prepared in a sterile environment after preliminary harvest (e.g., with a saw) and before being loaded into a growth chamber.

In a first condition, depleted substrate was rehydrated with enough water to be hydrated to 63% moisture content (e.g., as described above) and reground. One tool under these conditions resulted in the growth of a 224 g panel, which was 20.8% by wet mass as compared to a control panel (i.e., a panel grown from fresh substrate) (1078 g) and which was 20.9% by wet mass as compared to the panel previously grown from the same substrate (i.e., the “parent run,” before becoming depleted and rehydrated substrate). A second tool under the same conditions resulted in the growth of a 232 g panel, which was 21.5% by wet mass as compared to a control panel (i.e., a panel grown from fresh substrate) and which was 20.8% by wet mass as compared to the parent run (i.e., compared to the panel previously grown from the same substrate before becoming depleted and rehydrated substrate). Growth under these conditions may be described as wispy to dense, soggy, bulbous, and/or easily coming apart. Representative images of this growth are presented as FIGS. 7A and 7B.

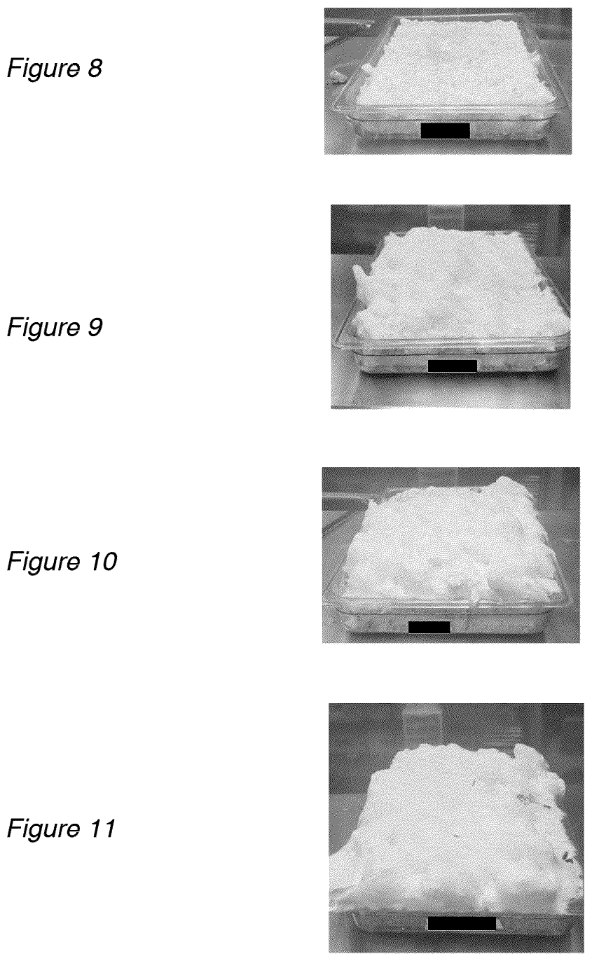

In a second condition, the top transitional layer of the depleted substrate was removed. This tool elicited no measurable growth (see FIG. 8). Growth under these conditions may be described as “no growth,” although some small dense protrusions or a small amount of wispy growth may be observed.

In a third condition, a tool was treated by removing the top transitional layer, gouging the top surface of the depleted substrate layer with a sturdy pipette tip (e.g., like “tilling”), and rehydrating. Under these conditions, the tool grew 104 g of aerial mycelia, which was 10.2% by wet mass as compared to the parent run (i.e., compared to the panel previously grown from the same substrate before becoming depleted and rehydrated substrate) and which was 9.6% by wet mass as compared to the control panel (i.e., a panel grown from fresh substrate). A representative image of this growth is presented as FIG. 9. Growth under these conditions may be described as wet tissue, wispy, strongly myceliated, and/or with gelatinous material towards the bottom of the tool.

In a fourth condition, a tool was treated by removing the top transitional layer, gouging the top surface of the depleted substrate layer with a sturdy pipette tip (e.g., like “tilling”), and rehydrating. Subsequently, the tool was inoculated with 183 g of grain inoculum across the top surface of the substrate layer. Under these conditions, the tool grew 114 g of aerial mycelia, which was 10.5% by wet mass as compared to the control panel (i.e., a panel grown from fresh substrate) and which was 11.2% by wet mass as compared to the parent run (i.e., compared to the panel previously grown from the same substrate before becoming depleted and rehydrated substrate). A representative image of this growth is presented as FIG. 10. Growth under these conditions may be described as soggy tissue.

In a fifth condition, a tool was treated by manually regrinding depleted substrate, reinoculating the depleted substrate with 190 g of grain inoculum, and rehydrating before loading the tool into a growth chamber. Under these conditions, the tool grew 372 g of aerial mycelia, which was 34.5% by wet mass as compared to the control panel (i.e., a panel grown from fresh substrate) and 36.7% by wet mass as compared to the parent run (i.e., compared to the panel previously grown from the same substrate before becoming depleted and rehydrated substrate). A representative image of this growth is presented as FIG. 11. Growth under these conditions may be described as wet, bulbous, with honeycomb-like features on the upper surface (i.e., deviant morphology), and/or with slimy exudate near the bottom of the tool.

Experiment 4

In another embodiment, charcoal powder can be added to the depleted substrate for further use. Charcoal powder as an additive can be effective for minimizing unfavorable effects of waste products exuded by mycelial tissue during growth. Through the first 13-day aerial mycelium growth cycle, no significant differences were observed in wet mass yield between the untreated and charcoal-treated substrate sets.

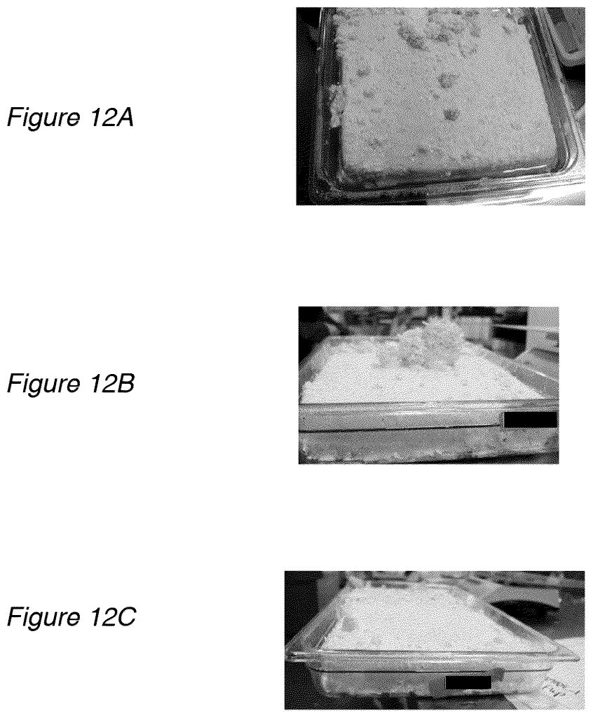

In a first treatment, the substrate block was flipped upside down so that the surface that had been against the bottom of the tool faced upwards. Of four replicates of this treatment (two with charcoal powder treated substrate, and two without), one exhibited 78 g of growth, which was 7.7% by wet mass as compared to a control panel (i.e., a panel grown from fresh substrate). Representative images of this growth are presented as FIGS. 12A-C. Growth under these conditions may be described as a ball of stipes, cauliflower-like, and/or substrate encased in an outer mycelial layer. The remaining three replicates that did not grow may be described as including yellow liquid which has collected within the container, with small grayish cauliflower-like primordia, and/or with a hard inner mycelia block.

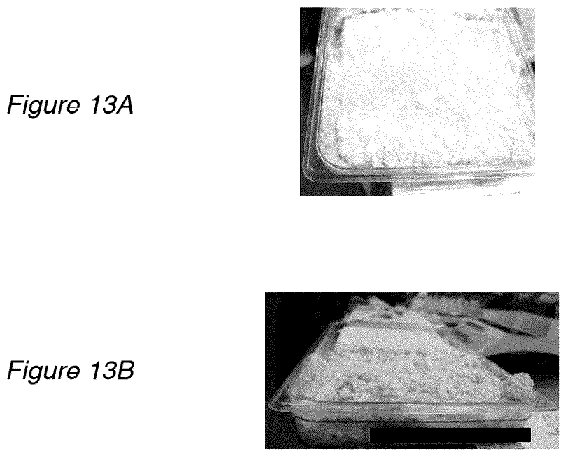

In a second treatment, the top ¼″ depth of the substrate (including the transitional layer) was removed from the harvested block. No further treatment was applied. None of the four replicates resulted in any measurable growth, meaning there was a 0% by wet mass yield when compared to the parent run (i.e., compared to the panel previously grown from the same substrate before becoming depleted and rehydrated substrate) or to the control panel (i.e., a panel grown from fresh substrate). Representative images of this growth are presented as FIGS. 13A and 13B. Growth under these conditions may be described as wispy, with yellow exudate, with grayish primordia, comprising small thin tendrils that grow into a mycelial mat, and/or webbed growth.

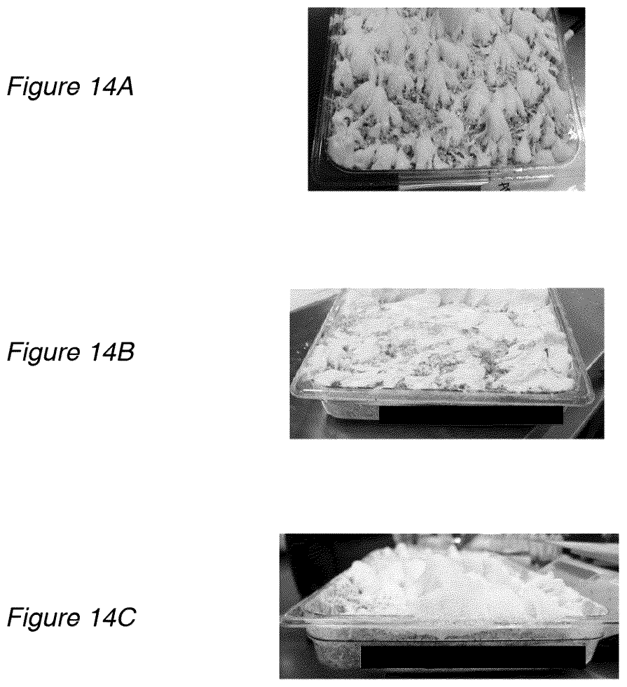

In a third treatment, the depleted substrate was removed, manually reground, and rehydrated to 65% after moisture content reading. Replicates of this treatment yielded 0 g, 88 g, 0 g and 0 g of mycelia. Representative images of this growth are presented as FIGS. 14A-C. Replicates that did not yield significant mycelial growth under these conditions may be described as wet, wispy, with short aerial extensions, with grayish primordia, poorly colonized, web-like growth, and/or with small bulbs.

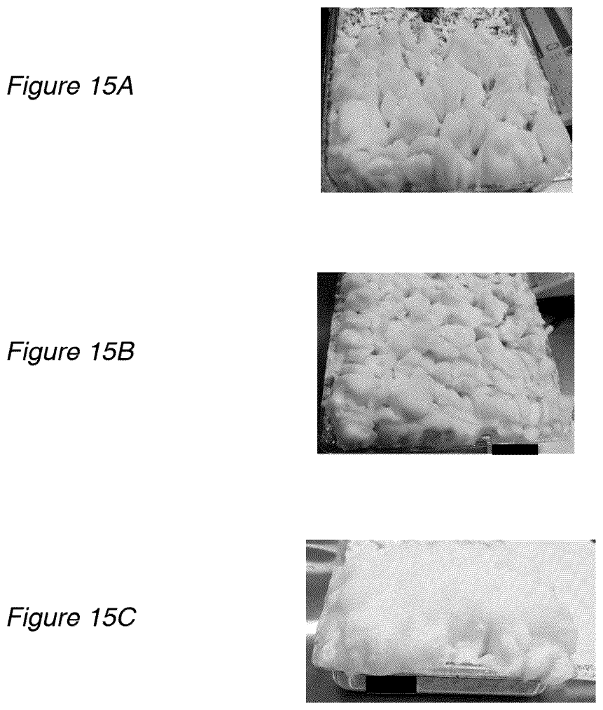

In a fourth treatment, depleted substrate was mixed with substrate blend (e.g., oak pellet and soybean hull pellet hydrated to 65% and sterilized) in a 1:1 ratio. This treatment yielded tissue that weighed an average of 112 g, with a minimum yield of no measurable growth and a maximum yield of 158 g, or 15.6% by wet mass as compared to a control panel (i.e., a panel grown from fresh substrate) and 20.5% by wet mass as compared to the parent run (i.e., compared to the panel previously grown from the same substrate before becoming depleted and rehydrated substrate). Representative images of this growth are presented as FIGS. 15A-C. Growth under these conditions may be described as tall, wispy, marshmallow-like, wet and/or slimy.

TABLE II
Experimental Support Summary for Experiment Nos. 5-6

| | | | Experiment |
|---|---|---|---|
| Status | Treatment | Outcome Compared to Control | No. |
| Resulted in | 50% precol, 50% | Comparable BE, 69% wet | 5 |
| equivalent or | depleted precol | yield, 83% dry yield | |
| increased mean | 75% precol, 25% | Comparable BE, 82% wet | 5 |
| bioefficiency | depleted precol | yield, comparable dry yield | |
| (BE) compared | | | |
| to control | | | |
| Resulted in | 100% sterilized depleted | ~50% BE, 54% wet yield, 52% | 5 |
| decreased mean | precol, reinoculated at | dry yield (compared to fresh | |
| BE compared to | 22% immediately before | control) | |
| control | use | | |
| | 25% precol, 75% | 58% dry BE, 42% wet BE, | 5 |
| | depleted precol | 50% wet yield, 53% dry yield | |
| | 100% depleted precol | 7.5% dry BE, 10% wet BE, 8% | 6 |
| | | wet and dry yield | |
| | 100% depleted precol, | 14% dry BE, 16% wet BE, | 6 |
| | inoculated at 22% | 15% dry yield, 18% wet yield | |
| | 100% depleted precol, | ~14% dry BE, ~15% wet BE, | 6 |
| | inoculated at 7% either | ~12% dry yield, ~16% wet | |
| | immediately before use | yield | |
| | or 4 days before use | | |
| | 50% depleted precol, | ~27% dry BE, ~38% wet BE, | 6 |
| | 50% fresh oak and soy | ~30% dry yield, ~40% wet | |
| | | yield | |
| | 99.9% depleted precol, | ~3% dry and wet BE, 3% dry | 6 |
| | 0.1% charcoal | yield, 7% wet yield | |

Experiment 5

In some embodiments, depleted precolonized substrate can be reinoculated with new precolonized substrate. Precolonized substrate refers to a nutritional growth matrix that is cooked, hydrated to a desired moisture content, inoculated with fungal spawn, and grown for a period of time (e.g., 4 days) before being used to procure aerial mycelial growth. Precolonized substrate which has been inoculated with fungal spawn and grown for 4 days before use may be referred to herein as “precol.” Precolonized substrate which has been inoculated with fungal spawn immediately before use may be referred to herein as “fresh precol.” “Depleted precol” refers to precol which has been used in a growth run and has been recovered after mycelial tissue harvesting, thus containing fewer nutrients than unused precol. Unless otherwise noted, depleted precol has been thoroughly broken up (e.g., to increase surface area) and rehydrated to about 65% moisture content before further use as precol. Without being bound by theory, the breaking up of depleted precol can have several advantages which may or may not directly relate to increased surface area, such as greater, easier, or faster gas exchange or rehydration, and/or stimulating aerial mycelial growth (e.g., faster and/or greater volume of aerial mycelial growth therefrom).

This experiment included 6 treatment groups (4 experimental, 2 control) packed into 40 mL tubes and grown in a growth chamber. Depleted precol was broken up and rehydrated to 65% moisture content before use. Treatments were as follows:
1. “Fresh Control”—Fresh Oak/Soy, inoculated at 22% immediately before use
2. “Precol Control”—100% precol
3. 50% precol, 50% depleted precol
4. 75% precol, 25% depleted precol
5. 25% precol, 75% depleted precol
6. 100% sterilized depleted precol, reinoculated at 22% immediately before use.

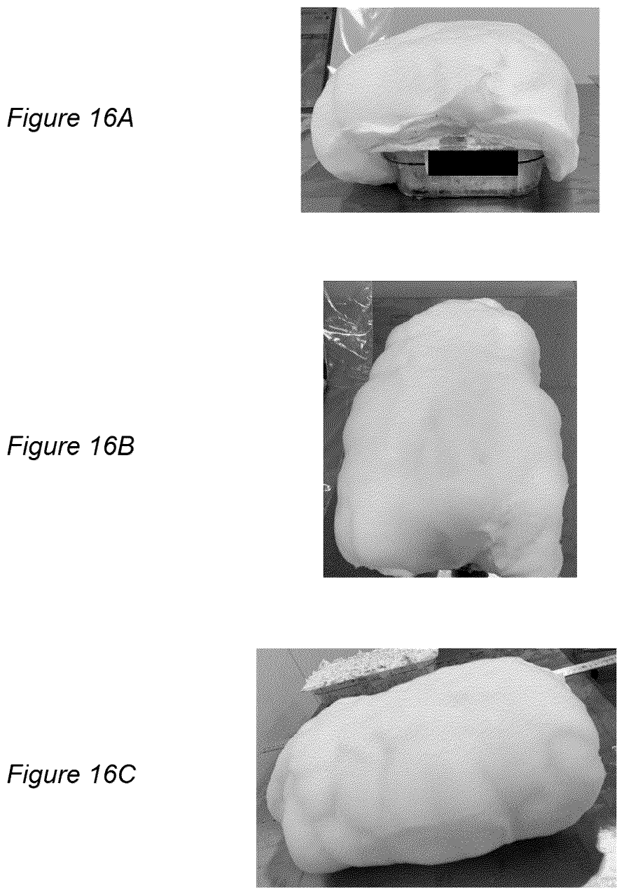

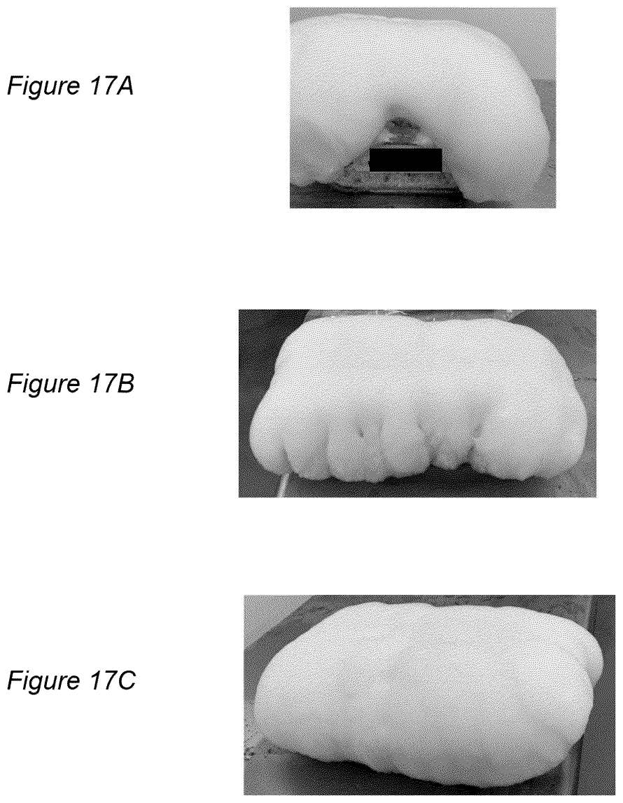

As used herein, bioefficiency (BE) refers to a measure of the efficiency with which substrate is converted to aerial mycelium, calculated by dividing the wet weight of aerial mycelium by the wet weight of substrate used to produce it (wet BE), or performing the same calculation using the respective dry weight measurements (dry BE). Mean wet BE and dry BE for treatments 3 and 4 were comparable to that of the precol control (treatment 2). Wet and dry mass yields were also similar to the precol control (treatment 2). Treatment 5 performed poorly compared to the other treatments. Treatment 6 yielded approximately 50% lower BE, wet mass yield, and dry mass yield compared to the fresh control (treatment 1). Representative images of growth under treatment 2 are presented as FIGS. 16A-C and representative images of growth under treatment 3 are presented as FIGS. 17A-C. These data indicate that supplementing depleted precol with new precol at a rate of 1:1 could have minimal impact on yield.

Experiment 6

In an additional example, various uses of depleted precol were tested. Nine treatment groups (7 experimental, 2 controls) were packed into 40 mL tubes and grown in a growth chamber. As described above in relation to Experiment 5, depleted precol was broken up and rehydrated to 65% moisture content before use. Precol treatment groups were prepared 4 days prior to packing and loading and were allowed to precolonize at room temperature. Treatments were as follows:
1. “Fresh Control”—Fresh Oak/Soy, inoculated at 22% immediately before use
2. “Precol Control”—100% precol
3. 100% depleted precol
4. 100% depleted precol, reinoculated at 22% immediately before use
5. 100% depleted precol, reinoculated at 7% immediately before use
6. 100% depleted precol, reinoculated at 7% 4 days before use
7. 50% depleted precol, 50% fresh uninoculated Oak/Soy
8. 50% depleted precol, 50% fresh uninoculated Oak/Soy, prepared 4 days before use
9. 99.9% depleted precol, 0.1% charcoal.

No treatment group resulted in dry BE, wet BE, dry mass yield, or wet mass yields that were similar in quantity as compared to control treatments. Comparing conditions involving reinoculated precol, treatments wherein the precol was reinoculated 4 days before use performed slightly better than treatments wherein the precol was reinoculated immediately before use, and all conditions yielded some aerial mycelium, but none of the yields involving reinoculated precol approached the yields of the control groups.

### Prophetic Example 7

Any number of ratio-mixtures of unused substrate to precolonized substrate (e.g., precol, fresh precol, or depleted precol) are contemplated herein, such as, e.g., 3:1, 2:1, 1:1, 1:2 and 1:3, or any ranges therebetween, wherein each ratio can represent a mass ratio of unused substrate to precol, unused substrate to fresh precol, unused substrate to depleted precol, or unused substrate to a combination of precol, fresh precol, and/or depleted precol.

### Embodiments

Embodiment 1: In some embodiments, a method of growing an aerial mycelium material can include providing a depleted growth matrix; applying a mechanical force to the depleted growth matrix; and disrupting the depleted growth matrix with the mechanical force. As an alternative to this embodiment, the mechanical force may be applied in either an active or passive manner. For an active manner, it may be by the application of manual or machine-movements acting upon the depleted growth matrix. For a passive manner, it may be by the weight of the depleted matrix being acted upon by gravity as the depleted matrix is positioned across a spatial gap or on an inclined slope during production. In a further alternative embodiment, once the depleted growth matrix has been disrupted, it may be directed either directly for use in the growth of aerial mycelium without further supplementation or rejuvenation, or alternatively it may be directed for use in the growth of mushrooms. In a further alternative embodiment, it may be directed to a rejuvenation step or steps, and/or reinoculated and then directed for use in the growth of aerial mycelium or mushrooms.

Embodiment 2: In some embodiments such as Embodiment 1, disrupting can include increasing the surface area of the depleted growth matrix.

Embodiment 3: In some embodiments such as Embodiment 1 or 2, disrupting can include at least one of grinding, breaking, fracturing, cutting, perforating, fragmenting, and physically manipulating the depleted growth matrix, such as for example by movement of the depleted growth matrix across a spatial gap or angled surface in production, whereby gravity is used to act upon the growth matrix to fracture it and break it apart.

In some embodiments such as any one of Embodiments 1 to 3, the method can include growing extra-particle aerial mycelial growth from the depleted growth matrix.

Embodiment 4: In some embodiments such as any one of Embodiments 1 to 3, the method can include rejuvenating at least a portion of the depleted growth matrix to form a rejuvenated growth matrix; and growing extra-particle aerial mycelial growth from the rejuvenated growth matrix.

In some embodiments such as any one of Embodiments 1 to 3, the method can include rejuvenating at least a portion of the depleted growth matrix to form a rejuvenated growth matrix; and growing one or more mushroom fruiting bodies from the rejuvenated growth matrix (under specific mushroom-favoring growth conditions).

Embodiment 5: In some embodiments such as Embodiment 4, the method can further include detaching the extra-particle aerial mycelial growth from the rejuvenated growth matrix to form a separated aerial mycelium from the extra-particle aerial mycelial growth, wherein the extra-particle aerial mycelial growth and the separated aerial mycelium do not include a fruiting body.

Embodiment 6: In some embodiments such as Embodiment 4 or 5, rejuvenating can include at least one of sterilizing the depleted growth matrix; rehydrating the depleted growth matrix; and supplementing the depleted growth matrix with an additive(s).

Embodiment 7: In some embodiments such as Embodiment 6, rejuvenating can include sterilizing the depleted growth matrix.

Embodiment 8: In some embodiments such as Embodiment 6, rejuvenating can include rehydrating the depleted growth matrix.

Embodiment 9: In some embodiments such as Embodiment 6, rejuvenating can include supplementing the depleted growth matrix with an additive(s).

Embodiment 10: In some embodiments such as Embodiment 9, rejuvenating can include supplementing the depleted growth matrix with the additive(s), wherein the additive(s) can include at least one of fresh fungal inoculum, fresh substrate or additional substrate, precolonized substrate, precolonized substrate which has been inoculated with fungal spawn and grown for up to 4 days before use, charcoal, and a nutrient source(s).

In some embodiments such as Embodiment 10, rejuvenating can include supplementing the depleted growth matrix with the additive(s) including fresh substrate at a mass ratio of between 1:3 and 3:1 (or any range contained therein) depleted growth matrix to fresh substrate.

Embodiment 11: In some embodiments such as Embodiment 10, the method can include providing a depleted growth matrix; applying a mechanical force to the depleted growth matrix; disrupting the depleted growth matrix with the mechanical force; rejuvenating at least a portion of the depleted growth matrix to form a rejuvenated growth matrix, wherein rejuvenating can include supplementing the depleted growth matrix with an additive(s), and wherein the additive(s) can include fresh substrate; and growing extra-particle aerial mycelial growth from the rejuvenated growth matrix.

Embodiment 12: In some embodiments, a method of growing an aerial mycelium material can include providing a first extra-particle aerial mycelial growth and a first growth matrix, wherein the first extra-particle aerial mycelial growth extends from a surface of the first growth matrix; dividing the first extra-particle aerial mycelial growth from the first growth matrix along a first separation zone to form a first separated aerial mycelium and a depleted growth matrix; rejuvenating at least a portion of the depleted growth matrix to form a rejuvenated growth matrix; and growing a second extra-particle aerial mycelial growth from the rejuvenated growth matrix.

Embodiment 13: In some embodiments such as Embodiment 12, the method can further include applying a mechanical force to the depleted growth matrix; and disrupting the depleted growth matrix with the mechanical force.

Embodiment 14: In some embodiments such as Embodiment 12 or 13, dividing can include dividing the first extra-particle aerial mycelial growth from the first growth matrix along the first separation zone such that the depleted growth matrix includes a transitional layer of unused extra-particle aerial mycelial growth on an underlying remainder portion of the depleted growth matrix.

Embodiment 15: In some embodiments such as Embodiment 14, the method can further include removing the transitional layer from the underlying remainder portion of the depleted growth matrix prior to disrupting the depleted growth matrix and growing the second extra-particle aerial mycelial growth.

In some embodiments such as Embodiment 14, the method can further include disrupting the depleted growth matrix, wherein the depleted growth matrix includes the transitional layer of unused extra-particle aerial mycelial growth on the underlying remainder portion of the depleted growth matrix.

Embodiment 16: In some embodiments such as any one of Embodiments 8 to 15, the first extra-particle aerial mycelial growth, the first separated aerial mycelium, and the second extra-particle aerial mycelial growth can not include a fruiting body.

Embodiment 17: In some embodiments such as any one of Embodiments 8 to 16, disrupting can include at least one of grinding, breaking, fracturing, cutting, perforating, fragmenting, and physical manipulating the depleted growth matrix. Such physical manipulation can include movement of the depleted growth matrix across a spatial divide, gap or angled surface whereby gravity can act on the depleted growth matrix, causing it to fracture and break apart under its own weight.

Embodiment 18: In some embodiments such as any one of Embodiments 8 to 16, rejuvenating can include at least one of sterilizing the depleted growth matrix; rehydrating the depleted growth matrix; and supplementing the depleted growth matrix with an additive(s).

Embodiment 19: In some embodiments such as Embodiment 18, rejuvenating can include sterilizing the depleted growth matrix.

Embodiment 20: In some embodiments such Embodiment 18, rejuvenating can include rehydrating the depleted growth matrix.

Embodiment 21: In some embodiments such as Embodiment 20, rehydrating the depleted growth matrix can include raising the moisture content of the depleted growth matrix to between about 60% to about 75%, alternatively between about 62% to about 75%, or alternatively between about 65% to about 75%.

Embodiment 22: In some embodiments such as Embodiment 20 or 21, rehydrating can further include gouging the substrate surface.

Embodiment 23: In some embodiments such as Embodiment 18, rejuvenating can include supplementing the depleted growth matrix with an additive(s).

Embodiment 24: In some embodiments such as Embodiment 23, the additive(s) can include at least one of fresh fungal inoculum, fresh substrate, precolonized substrate, precolonized substrate which has been inoculated with fungal spawn and grown for up to 4 days before use, charcoal, and a nutrient source(s).

In some embodiments such as Embodiment 24, rejuvenating can include supplementing the depleted growth matrix with the additive(s) comprising fresh substrate at a mass ratio of between 1:3 and 3:1 (or any range therebetween) depleted growth matrix to fresh substrate.

Embodiment 25: In some embodiments such as Embodiment 24, the first growth matrix can include a first fungal inoculum, wherein the additive(s) can include the fresh fungal inoculum, and wherein the first fungal inoculum and the fresh fungal inoculum can include a different species relative to each other.

Embodiment 26: In some embodiments such as any one of Embodiments 12 to 25, the method can further include dividing the second extra-particle aerial mycelial growth from the rejuvenated growth matrix along a second separation zone to form a second separated aerial mycelium and a second depleted growth matrix; and cutting the second separated aerial mycelium in a transverse direction and across a width of the second separated aerial mycelium to form an aerial mycelium panel.

Embodiment 27: In some embodiments, a method of growing an aerial mycelium material without production of fruiting bodies can include providing a depleted growth matrix; applying a mechanical force to the depleted growth matrix; disrupting the depleted growth matrix with the mechanical force; rejuvenating at least a portion of the first depleted growth matrix to form a rejuvenated growth matrix; and growing an extra-particle aerial mycelial growth from the rejuvenated growth matrix without producing any fruiting bodies.

Embodiment 28: In some embodiments, a method of making an aerial mycelium panel can include growing a first extra-particle aerial mycelial growth from a first growth matrix such that the first extra-particle aerial mycelial growth extends from a surface of the first growth matrix, and without producing any fruiting bodies; harvesting a first aerial mycelium panel from the first extra-particle aerial mycelial growth, wherein harvesting can include dividing the first extra-particle aerial mycelial growth from the first growth matrix along a first separation zone to form a first separated aerial mycelium and a first depleted growth matrix and cutting the first separated aerial mycelium in a transverse direction and across a width of the first separated aerial mycelium to form the first aerial mycelium panel; applying a mechanical force to the first depleted growth matrix; disrupting the first depleted growth matrix with the mechanical force; rejuvenating at least a portion of the first depleted growth matrix to form a rejuvenated growth matrix; growing a second extra-particle aerial mycelial growth from the rejuvenated growth matrix such that the second extra-particle aerial mycelial growth extends from a surface of the rejuvenated growth matrix, and without producing any fruiting bodies; and harvesting a second aerial mycelium panel from the second extra-particle aerial mycelial growth, wherein harvesting can include dividing the second extra-particle aerial mycelial growth from the rejuvenated growth matrix along a second separation zone to form a second separated aerial mycelium and a second depleted growth matrix and cutting the second separated aerial mycelium in a transverse direction and across a width of the second separated aerial mycelium to form the second aerial mycelium panel.

Embodiment 29: In some embodiments such as Embodiment 28, at least one of the first depleted growth matrix and the second depleted growth matrix can include a transitional layer of unused extra-particle aerial mycelial growth and an underlying remainder portion, wherein the method can further include removing the transitional layer from the underlying remainder portion.

Embodiment 30: In some embodiments such as Embodiment 29, the first separated aerial mycelium and the second separated aerial mycelium can be substantially the same.

Embodiment 31: In some embodiments such as any one of Embodiments 28 to 30, the first separated aerial mycelium and the second separated aerial mycelium can be substantially the same with respect to one or more of the following physical characteristics: elasticity, tensile strength, rigidity, density, shear strength, texture, and compressive strength.

Embodiment 32: In some embodiments such as Embodiment 31, the first separated aerial mycelium and the second separated aerial mycelium can be substantially the same with respect to two or more of the physical characteristics.

Embodiment 33: In some embodiments such as Embodiment 31, the first separated aerial mycelium and the second separated aerial mycelium can be substantially the same with respect to three or more of the physical characteristics.

Embodiment 34: In some embodiments such as Embodiment 31, the first separated aerial mycelium and the second separated aerial mycelium can be substantially the same with respect to all of the physical characteristics.

Embodiment 35: In some embodiments, a method of making an aerial mycelium panel can include growing a first extra-particle aerial mycelial growth from a first growth matrix such that the first extra-particle aerial mycelial growth extends from a surface of the first growth matrix, and without producing any fruiting bodies; harvesting a first aerial mycelium panel from the first extra-particle aerial mycelial growth, wherein harvesting can include dividing the first extra-particle aerial mycelial growth from the first growth matrix along a first separation zone to form a first separated aerial mycelium and a first depleted growth matrix and cutting the first separated aerial mycelium in a transverse direction and across a width of the first separated aerial mycelium to form the first aerial mycelium panel; applying a mechanical force to the first depleted growth matrix; disrupting the first depleted growth matrix with the mechanical force; rejuvenating at least a portion of the first depleted growth matrix to form a rejuvenated growth matrix; growing a second extra-particle aerial mycelial growth from the rejuvenated growth matrix such that the second extra-particle aerial mycelial growth extends from a surface of the rejuvenated growth matrix, and without producing any fruiting bodies; and harvesting a second aerial mycelium panel from the second extra-particle aerial mycelial growth, wherein harvesting can include dividing the second extra-particle aerial mycelial growth from the rejuvenated growth matrix along a second separation zone to form a second separated aerial mycelium and a second depleted growth matrix and cutting the second separated aerial mycelium in a transverse direction and across a width of the second separated aerial mycelium to form the second aerial mycelium panel, and further wherein said second aerial mycelium panel differs from said first aerial mycelium panel in at least one of the following physical characteristics selected from the group consisting of elasticity, tensile strength, rigidity, density, shear strength, texture, and compressive strength.

Embodiment 36: In some embodiments, an apparatus for separating and rejuvenating a depleted growth matrix can include a separation component for processing extra-particle aerial mycelial growth and a growth matrix, comprising dividing means to divide and detach the extra-particle aerial mycelial growth from the growth matrix and form an aerial mycelium and a depleted growth matrix, an optional cutting means to cut the aerial mycelium and form an aerial mycelium panel, and diverter means to contain the depleted growth matrix and to divert the depleted growth matrix away from the aerial mycelium or the aerial mycelium panel; a disrupting component for exposing an increased surface area of said depleted growth matrix, wherein said disrupting component can be in direct transport communication with said separation component, and wherein said disrupting component can include at least one of a surface topography altering means and a particulate breaking means; and an optional inoculum-adding, rehydration-adding, and/or additive(s)-adding component in direct transport communication with said disrupting component.

Embodiment 37: In some embodiments, a method of growing either aerial mycelium material or mushrooms can include providing a depleted growth matrix which has been depleted by a previous growth of mycelium therein and/or thereupon; applying a mechanical force to the depleted growth matrix, thereby disrupting the depleted growth matrix with the mechanical force; and then growing either aerial mycelium or mushrooms upon the depleted growth matrix (under growth conditions appropriately tailored to then preferentially foster either the growth of aerial mycelium or mushrooms). In some further embodiments, the method includes at least one of the following conditions: (i) the previous growth of mycelium includes growth conditions preferentially favoring the growth of predominantly aerial mycelium, and (ii) the growing of either aerial mycelium or mushrooms upon the depleted growth matrix includes growing aerial mycelium under growth conditions preferentially favoring the growth of predominantly aerial mycelium.

Embodiment 38: In some embodiments, a method of growing either aerial mycelium material or mushrooms can include providing a depleted growth matrix which has been depleted by a previous growth of mycelium therein and/or thereupon; applying a mechanical force to the depleted growth matrix, thereby disrupting the depleted growth matrix with the mechanical force; and then growing either aerial mycelium or mushrooms upon the depleted growth matrix, wherein at least one of the following conditions is met: (i) the previous growth of mycelium includes growth conditions preferentially favoring the growth of predominantly aerial mycelium, and (ii) the growing of either aerial mycelium or mushrooms upon the depleted growth matrix includes growing aerial mycelium under growth conditions preferentially favoring the growth of predominantly aerial mycelium.

Embodiment 39: In some embodiments such as Embodiment 37 or 38, the previous growth of mycelium can have been for predominantly or solely aerial mycelium material production (under growth conditions that were appropriately tailored to preferentially foster the growth of aerial mycelium). In alternative embodiments of Embodiment 37 or 38, the previous growth of mycelium (as opposed to aerial mycelium) can have been for the growth of predominantly or entirely mushroom fruiting bodies (as opposed to aerial mycelium, and under growth conditions appropriately tailored to preferentially foster the growth of mushrooms).

Embodiment 40: In some embodiments such as any one of Embodiments 37 to 39, growing either aerial mycelium or mushrooms upon the depleted growth matrix can include growing predominantly aerial mycelium upon the depleted growth matrix. In alternative embodiments of any one of Embodiments 37 to 39, growing either aerial mycelium or mushrooms upon the depleted growth matrix can include growing predominantly mushroom fruiting bodies upon the depleted growth matrix.

Embodiment 41: In some embodiments such as any one of Embodiments 37 to 40, the method can include rejuvenating the depleted growth matrix following the disrupting.

Embodiment 42: In some embodiments such as any one of Embodiments 37 to 41, the method can include containing the depleted growth matrix within packaging following the disrupting for later growth of mushrooms.

Embodiment 43: In some embodiments such as any one of Embodiments 37 to 42, the previous growth of mycelium can result in aerial mycelium without any mushroom fruiting bodies, wherein the growing of either aerial mycelium or mushrooms upon the depleted growth matrix can include growing one or more mushroom fruiting bodies.

Embodiment 44: In some embodiments such as any one of Embodiments 37 to 42, the previous growth of mycelium can result in mycelium with one or more mushroom fruiting bodies, wherein the growing of either aerial mycelium or mushrooms upon the depleted growth matrix can include growing aerial mycelium without any mushroom fruiting bodies.

Embodiment 45: In some embodiments, a method of growing either aerial mycelium material or mushrooms can include forming a depleted growth matrix by at least one of (i) growing and harvesting aerial mycelium from an aerial mycelium growth matrix without producing any fruiting bodies and (ii) growing and harvesting one or more mushrooms bodies from a mushroom growth matrix, applying a mechanical force to the depleted growth matrix thereby disrupting the depleted growth matrix with the mechanical force, and growing either aerial mycelium or mushrooms upon the depleted growth matrix, wherein at least one of the following conditions is met: (a) the forming of the depleted growth matrix includes growing and harvesting aerial mycelium from the aerial mycelium growth matrix without producing any fruiting bodies, and (b) the growing either aerial mycelium or mushrooms upon the depleted growth matrix includes growing aerial mycelium upon the depleted growth matrix.

Scope of Disclosure

While certain embodiments of the inventions have been described, these embodiments have been presented by way of example only and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings) and/or all of the steps of any method or process so disclosed may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings) or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Although features may be described above as acting in certain combinations, one or more features from a claimed combination can, in some cases, be excised from the combination, and the combination may be claimed as a subcombination or variation of a subcombination.

The features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately,” “about,” “generally,” and “substantially” may refer to an amount that is within less than 10% of, within less than 5% of, within less than 1% of, within less than 0.1% of, and within less than 0.01% of the stated amount.

The scope of the present disclosure is not intended to be limited by the specific disclosures of embodiments in this section or elsewhere in this specification and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. A method of growing an aerial mycelium material comprising:
providing a depleted growth matrix;
applying a mechanical force to the depleted growth matrix; and
disrupting the depleted growth matrix with the mechanical force.

2. The method of claim 1, wherein disrupting comprises at least one of grinding, breaking, fracturing, cutting, perforating, fragmenting, and physically manipulating the depleted growth matrix.

3. The method of claim 1, further comprising:
growing extra-particle aerial mycelial growth from the depleted growth matrix.

4. The method of claim 1, further comprising:
rejuvenating at least a portion of the depleted growth matrix to form a rejuvenated growth matrix; and
growing extra-particle aerial mycelial growth from the rejuvenated growth matrix.

5. The method of claim 1, further comprising:
rejuvenating at least a portion of the depleted growth matrix to form a rejuvenated growth matrix; and
growing one or more mushroom fruiting bodies from the rejuvenated growth matrix.

6. The method of claim 4, further comprising detaching the extra-particle aerial mycelial growth from the rejuvenated growth matrix to form a separated aerial mycelium from the extra-particle aerial mycelial growth, wherein the extra-particle aerial mycelial growth and the separated aerial mycelium do not comprise a fruiting body.

7. The method of claim 4, wherein rejuvenating comprises at least one of:
sterilizing the depleted growth matrix;
rehydrating the depleted growth matrix; and
supplementing the depleted growth matrix with an additive(s).

8. The method of claim 7, wherein rejuvenating comprises supplementing the depleted growth matrix with the additive(s), and wherein the additive(s) comprises at least one of: fresh fungal inoculum, fresh substrate, precolonized substrate, precolonized substrate which has been inoculated with fungal spawn and grown for up to 4 days before use, charcoal, and a nutrient source(s).

9. The method of claim 8, wherein rejuvenating comprises supplementing the depleted growth matrix with the additive(s) comprising fresh substrate at a mass ratio of between 1:3 and 3:1 depleted growth matrix to fresh substrate.

10. The method of claim 8, wherein the method comprises:
providing the depleted growth matrix;
applying the mechanical force to the depleted growth matrix;
disrupting the depleted growth matrix with the mechanical force;
rejuvenating the at least portion of the depleted growth matrix to form the rejuvenated growth matrix,
wherein rejuvenating comprises supplementing the depleted growth matrix with the additive(s), and
wherein the additive(s) comprises the fresh substrate; and
growing extra-particle aerial mycelial growth from the rejuvenated growth matrix.

11. A method of growing an aerial mycelium material comprising:
providing a first extra-particle aerial mycelial growth and a first growth matrix, wherein the first extra-particle aerial mycelial growth extends from a surface of the first growth matrix;
dividing the first extra-particle aerial mycelial growth from the first growth matrix along a first separation zone to form a first separated aerial mycelium and a depleted growth matrix;
rejuvenating at least a portion of the depleted growth matrix to form a rejuvenated growth matrix; and
growing a second extra-particle aerial mycelial growth from the rejuvenated growth matrix.

12. The method of claim 11, further comprising:
applying a mechanical force to the depleted growth matrix; and
disrupting the depleted growth matrix with the mechanical force.

13. The method of claim 12, wherein disrupting comprises at least one of grinding, breaking, fracturing, cutting, perforating, fragmenting, and physically manipulating the depleted growth matrix.

14. The method of claim 11, wherein dividing comprises dividing the first extra-particle aerial mycelial growth from the first growth matrix along the first separation zone such that the depleted growth matrix includes a transitional layer of unused extra-particle aerial mycelial growth on an underlying remainder portion of the depleted growth matrix.

15. The method of claim 14, further comprising removing the transitional layer from the underlying remainder portion of the depleted growth matrix prior to disrupting the depleted growth matrix and growing the second extra-particle aerial mycelial growth.

16. The method of claim 11, wherein rejuvenating comprises at least one of sterilizing the depleted growth matrix, rehydrating the depleted growth matrix, and supplementing the depleted growth matrix with an additive(s).

17. The method of claim 16, wherein rejuvenating comprises rehydrating the depleted growth matrix by raising the moisture content of the depleted growth matrix to between about 60% to about 75%, alternatively between about 62% to about 75%, or alternatively between about 65% to about 75%.

18. The method of claim 16, wherein rehydrating further comprises gouging the substrate surface.

19. The method of claim 16, wherein rejuvenating comprises supplementing the depleted growth matrix with the additive(s), and wherein the additive(s) comprises at least one of: fresh fungal inoculum, fresh substrate, precolonized substrate, precolonized substrate which has been inoculated with fungal spawn and grown for up to 4 days before use, charcoal, and a nutrient source(s).

20. The method of claim 19, wherein rejuvenating comprises supplementing the depleted growth matrix with the additive(s) comprising fresh substrate at a mass ratio of between 1:3 and 3:1 depleted growth matrix to fresh substrate.

21. The method of claim 19, wherein the first growth matrix comprises a first fungal inoculum, and wherein the additive(s) comprises the fresh fungal inoculum, wherein the first fungal inoculum and the fresh fungal inoculum comprise a different species relative to each other.

22. The method of claim 11, further comprising:
dividing the second extra-particle aerial mycelial growth from the rejuvenated growth matrix along a second separation zone to form a second separated aerial mycelium and a second depleted growth matrix; and
cutting the second separated aerial mycelium in a transverse direction and across a width of the second separated aerial mycelium to form an aerial mycelium panel.

23-31. (canceled)
